SEQUENCE LISTING (6 sequences)

SEQ ID NO: 1 - protein, 12 aa, synthetic construct
HHIYLGAVNYIY

SEQ ID NO: 2 - protein, 12 aa, synthetic construct
YIYNVAGLYIHH

SEQ ID NO: 3 - protein, 12 aa, synthetic construct
  SITE 3: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 4: note = Tyrosine or modified tyrosine
  SITE 10: note = Tyrosine or modified tyrosine
  SITE 11: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 12: note = Tyrosine or modified tyrosine
HHXXLGAVNXXX

SEQ ID NO: 4 - protein, 12 aa, synthetic construct
  SITE 1: note = Tyrosine or modified tyrosine
  SITE 2: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
  SITE 3: note = Tyrosine or modified tyrosine
  SITE 9: note = Tyrosine or modified tyrosine
  SITE 10: note = Any amino acid with a 1-propyl, 2-propyl, 2-methyl-prop-2-yl, C3-6-cycloalkyl, C4-6-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran- 4-yltetrahydrothiopyran-2-yl, tetrahydrothiopyran-3-yl, tetrahydrothiopyran-4-yl or 1-CH(OR5)CH3 side chain
XXXNVAGLXXHH

SEQ ID NO: 5 - protein, 12 aa, synthetic construct
YVYNVAGLYVHH

SEQ ID NO: 6 - protein, 12 aa, synthetic construct
  SITE 10: note = Histidine with C-terminal-1-Acetylpiperazine
YVYNVAGLYVHH

(19) United States
(12) Patent Application Publication
(10) Pub. No.: US 2026/0199424 A1
(43) Pub. Date: Jul. 16, 2026

(54) METHODS AND COMPOSITIONS FOR IMPROVING VISUAL FUNCTION IN OCULAR DISEASES AND DISORDERS

(71) Applicant: ONL Therapeutics, Inc., Ann Arbor, MI (US)

(72) Inventors: Andrew J. KOCAB, Farmington Hills, MI (US); David N. ZACKS, Ann Arbor, MI (US); Constance I. CHANG, Ann Arbor, MI (US); Lindsay M. PUSCAS, Northville, MI (US); Stephanie C. WIETHOLTER, Snellville, GA (US); Lori Lynn HUANG, Saline, MI (US); David A. ESPOSITO, Ann Arbor, MI (US); Sushanta MALLICK, Richardson, TX (US); Mitchell G. BRIGELL, Belmont, MA (US); David M. KLEINMAN, Rochester, NY (US); Jana VAN DE GOOR, Jerome, MI (US)

(21) Appl. No.: 19/136,693

(22) PCT Filed: Dec. 7, 2023
(86) PCT No.: PCT/US2023/082934

§ 371 Date: Jun. 6, 2025

Related U.S. Application Data
(60) Provisional application No. 63/386,852, filed on Dec. 9, 2022.
(60) Provisional application No. 63/386,854, filed on Dec. 9, 2022.
(60) Provisional application No. 63/587,388, filed on Oct. 2, 2023.
(60) Provisional application No. 63/601,416, filed on Nov. 21, 2023.

(51) Int. Cl. A61K 38/10; A61P 27/02; A61P 27/06

(52) U.S. Cl. CPC A61K 38/10; A61P 27/02; A61P 27/06

(57) ABSTRACT

Provided herein are methods and compositions for improving visual function in an eye of an individual having an ocular disorder. Provided herein are methods of improving visual function in an eye of an individual having an ocular disease or disorder, the method comprising: (a) administering a first composition comprising Fas inhibitor; and (b) administering a second composition comprising the Fas inhibitor, wherein the second composition is administered about 10 weeks or more after administering the first composition.

### CROSS REFERENCE

This application claims the benefit of U.S. Provisional Application No. 63/386,852 filed on Dec. 9, 2022, U.S. Provisional Application No. 63/386,854 filed on Dec. 9, 2022, U.S. Provisional Application No. 63/587,388 filed on Oct. 2, 2023, U.S. Provisional Application No. 63/601,416 filed on Nov. 21, 2023, each of which is incorporated by reference herein in its entirety.

### BACKGROUND

Vision is generally dependent on maintaining the anatomical and histological integrity of the structures of the eye. Changes in retinal homeostasis can provide the basis for vision loss and/or retinal diseases or disorders. Therapies for the treatment of ocular diseases and disorders generally target stopping or reducing the deleterious changes associated ocular dysfunction resulting from diseases or disorders of the eye.

### SUMMARY

Targeting a cessation or a reduction of deleterious changes to visual function associated with ocular disease can be useful in treatment programs, however, restoration and improvement of visual function represents a more preferred and, in general instances, more difficult to achieve patient outcome. Described and provided herein are compositions and methods useful for improving visual function in an eye of an individual having an ocular disease and/or disorder.

Provided herein are methods of improving visual function (e.g., BCVA or visual field) in an eye of an individual having an ocular disease or disorder, comprising: (a) administering a first composition comprising a peptide to the eye of the individual, the peptide comprising an amino acid sequence HHIYLGAVNYIY or a variant sequence thereof, or a pharmaceutically acceptable salt of the peptide; and (b) administering a second composition comprising the peptide to the eye, wherein the second composition is administered about 10 weeks or more after administering the first composition. Also provided are methods of improving visual function (e.g., BCVA or visual field) in an eye of an individual having an ocular ssdisease or disorder, comprising: (a) administering a first composition comprising a peptide to the eye of the individual, the peptide having the structure of Formula I or a pharmaceutically acceptable salt thereof; and (b) administering a second composition comprising the peptide to the eye, wherein the second composition is administered about 10 weeks or more after administering the first composition. Further provided are methods of improving visual function (e.g., BCVA or visual field) in an eye of an individual having an ocular disease or disorder, comprising: (a) administering a first composition comprising a peptide to the eye of the individual, the peptide having the structure of Formula III or a pharmaceutically acceptable salt thereof; and (b) administering a second composition comprising the peptide to the eye, wherein the second composition is administered about 10 weeks or more after administering the first composition.

In some embodiments, the second composition is administered about 8 weeks or more after administering the first composition. In some embodiments, the second composition is administered about 10 weeks or more after administering the first composition. In some embodiments, the second composition is administered about 12 weeks or more after administering the first composition. In some embodiments, the second composition is administered about 16 weeks or more after administering the first composition. In some embodiments, the second composition is administered about 20 weeks or more after administering the first composition. In some embodiments, the second composition is administered about 24 weeks or more after administering the first composition.

In some embodiments, the visual function is improved after administering the first composition, wherein administering the second composition maintains the improvement in visual function. In some embodiments, the visual function is improved after administering the second composition. In some embodiments, the visual function is improved after administering the first composition and is further improved after administering the second composition.

Further provided herein are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide comprising an amino acid sequence HHIYLGAVNYIY or variant sequence thereof, or a pharmaceutically acceptable salt thereof; and no greater than five compositions are administered to the eye within a year. Also provided are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide having the structure of Formula I or a pharmaceutically acceptable salt thereof; and no greater than five compositions are administered to the eye within a year. Additionally provided are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide having the structure of Formula III or a pharmaceutically acceptable salt thereof; and no greater than five compositions are administered to the eye within a year.

In some embodiments, no greater than four compositions are administered to the eye within a year. In some embodiments, no greater than three compositions are administered to the eye within a year. In some embodiments, no greater than two compositions are administered to the eye within a year. In some embodiments, the plurality of compositions comprises 2 compositions. In some embodiments, the plurality of compositions comprises 3 compositions. In some embodiments, the plurality of compositions comprises 4 compositions. In some embodiments, the plurality of compositions comprises 5 compositions.

In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

In some embodiments, the visual function is improved after administering a first composition, wherein administering subsequence compositions maintain the improvement in visual function. In some embodiments, the visual function is improved after administering multiple compositions (e.g., two or more). In some embodiments, the visual function is improved after administering a first composition and is further improved after administering an additional one (e.g., a second) or more (e.g., a third or fourth) composition.

In some embodiments, the method comprises administering each composition to the vitreous humor of the eye. In some embodiments, the peptide has a half-life in the vitreous humor greater than about 30 days. In some embodiments, the peptide has a half-life in the vitreous humor greater than about 90 days. In some embodiments, the peptide has a half-life in the vitreous humor greater than about 180 days. In some embodiments, the peptide has a half-life in the vitreous humor greater than about 200 days. In some embodiments, the method comprises using the vitreous humor as a depot to provide the peptide to retinal tissue in the eye.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows data demonstrating improved visual acuity outcomes in GA patients receiving a Fas inhibitor.

FIG. 2A-2B provides exemplary conversions of measures of visual acuity. FIG. 2A shows a first exemplary conversion table of measures of visual acuity. FIG. 2B shows a second exemplary conversion table of measures of visual acuity.

FIG. 3 shows cluster data for visual field outcomes in glaucoma patients receiving no Fas inhibitor.

FIG. 4 shows cluster data demonstrating improved visual field outcomes in glaucoma patients receiving multiple 50 microgram doses of a Fas inhibitor.

FIG. 5 shows cluster data demonstrating improved visual field outcomes in glaucoma patients receiving multiple 100 microgram doses of a Fas inhibitor.

FIG. 6 shows individual point data demonstrating improved visual field outcomes in glaucoma patients receiving multiple 50 microgram doses of a Fas inhibitor.

FIG. 7 shows individual point data demonstrating improved visual field outcomes in glaucoma patients receiving multiple 100 microgram doses of a Fas inhibitor.

FIG. 8 shows individual point data for visual field outcomes in glaucoma patients receiving no Fas inhibitor.

FIG. 9 shows data from glaucoma patients for historical visual field rate of loss/year against visual function cluster baseline sensitivity.

FIGS. 10A and 10B show cluster data from glaucoma patients for historical visual field rate of loss/year against visual field rate of change/year post treatment with a Fas inhibitor. Treatment with a Fas inhibitor generally improved the visual field rate of change/year relative to the sham control.

FIGS. 11A, 11B, and 11C show cluster data from glaucoma patients for visual function baseline sensitivities against visual field rate of change/year post treatment with a Fas inhibitor. Treatment with a Fas inhibitor generally improved and/or protected against a loss of the visual field rate of change/year relative to the sham control.

### DETAILED DESCRIPTION

Described and provided herein are compositions and methods useful for improving visual function in an eye of an individual having an ocular disease and/or disorder. In certain instances, the improvement of visual function is achieved by utilizing multiple doses of the Fas inhibitor. Retinal degeneration is a complex, multifactorial condition where cell death (e.g., cell apoptosis), glial cell activation, and inflammation in or surrounding retinal tissue are linked to the death of cells within the retina (e.g., retinal pigment epithelial (RPE) cells and/or photoreceptors (PRs)). Fas-mediated inflammation can directly or indirectly lead to retinal degeneration and can be associated with the symptoms resulting from retinal degeneration. The Fas-mediated inflammation signaling pathway is generally initiated by an interaction between the membrane-bound Fas ligand (FasL—a type II transmembrane protein of the TNF family) and Fas receptor, thereby leading to the activation of pro-inflammatory signaling (e.g., cytokine signaling, interleukin signaling, caspase action, etc.) and/or cell death signaling (e.g., apoptotic signaling, necrotic signaling, etc.) pathways.

### Fas Inhibitors

Provided herein are Fas inhibitors useful for modulating (e.g., inhibiting, preventing, and/or reducing, etc.) Fas-mediated signaling. In certain instances, the Fas inhibitors useful in treating, inhibiting, preventing, and/or reducing Fas-mediated inflammation. In certain instances, inhibiting, preventing, and/or reducing Fas-mediated inflammation allows for the treatment and/or prevention of cell loss within the retina (e.g., photoreceptors and/or retinal pigment epithelium). Accordingly, in certain instances, the Fas inhibitors are also useful in treating and/or preventing retinal cell loss and/or the symptoms associated with retinal cell loss. In certain instances, inhibiting, preventing, and/or reducing Fas-mediated inflammation further treats, inhibits, reduces, and/or prevents retinal cell loss (e.g., retinal epithelial (RPE) cells and/or photoreceptor (PR)). cells). In some embodiments, the Fas inhibitors described herein are useful for treating, inhibiting, reducing, and/or preventing retinal cell loss and/or the symptoms associated with retinal cell loss. In such instances, because the Fas inhibitors described herein are useful in the manner described above, the Fas inhibitors are useful in method of treating ocular diseases and disorders. In some embodiments, the Fas inhibitor is a peptide, a polypeptide (e.g., FasR or FasL), an antibody that binds FasR, or antibody that binds FasL.

In some embodiments, the Fas inhibitors described herein encompass Met-derived peptides and/or fragments thereof. In some embodiments, the Met protein, also called c-Met or hepatocyte growth factor receptor (HGF receptor), is encoded by the Met gene (NCBI Gene ID 4233, Location: NC_000007.14 (116672196 . . . 116798386), UniProtKB—P0858). The Met protein is comprised of two major subunits: the α and β subunits, and Met and fragments of Met, including the extracellular domain of Met and its a subunit, have been shown to bind to Fas and prevent cells from undergoing apoptosis. In some embodiments, the Fas inhibitor comprises a Fas-inhibiting peptide (e.g., Met-derived peptide and/or fragment thereof). In some embodiments, the Fas inhibitors described herein comprises a Met-derived compound comprising the amino acid acids HHIYLGAVNYIY (His-His-Ile-Tyr-Leu-Gly-Ala-Val-Asn-Tyr-Ile-Tyr) (e.g., SEQ ID NOs: 1-8). In some embodiments, the peptide comprises the amino acid sequence HHIYLGAVNYIY or a variant sequence thereof.

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the Fas inhibitor peptides (e.g., a peptide comprising the amino acid sequence HHIYLGAVNYIY) comprises one or more naturally occurring amino acids. In some embodiments, the Fas inhibitor peptides (e.g., a peptide comprising the amino acid sequence HHIYLGAVNYIY) consists of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the Fas inhibitor comprises a variant sequence of the peptide (e.g., a peptide comprising the amino acid sequence HHIYLGAVNYIY). In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, the variant sequence comprises one amino acid substitution. In some embodiments, the variant sequence comprises two amino acid substitutions. In some embodiments, the variant sequence comprises three amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In some embodiments, the Fas inhibitor peptides (e.g., a peptide comprising the amino acid sequence HHIYLGAVNYIY) comprises one or more non-natural amino acids. In some embodiments, the Fas inhibitor peptides (e.g., a peptide comprising the amino acid sequence HHIYLGAVNYIY) consists of non-natural amino acids. As used herein, non-natural amino acids and/or unnatural amino acids include and/or refer to amino acid structures that cannot be generated biosynthetically in any organism using unmodified or modified genes from any organism. In some embodiments, non-natural amino acids and/or unnatural amino acids further include and/or refer to an amino acid residue that are not present in the naturally occurring (wild-type) Met protein sequence. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring (wild-type) Met protein sequence. A non-natural amino acid residue also can be incorporated such that a desired functionality is imparted to the apelin molecule, for example, the ability to link a functional moiety (e.g., PEG).

In some embodiments, a variant sequence comprises one or more amino acid deletions. In some embodiments, the variant sequence comprises one amino acid deletion. In some embodiments, the variant sequence comprises two amino acid deletions. In some embodiments, the variant sequence comprises three amino acid deletions. In some embodiments, the variant sequence comprises four amino acid deletions. In some embodiments, the variant sequence comprises one or more additional amino acids. In some embodiments, the additional amino acids are additional amino acids from the Met sequence. In some embodiments, the variant sequence comprises a substitution and a deletion. In some embodiments, the variant sequence comprises a substitution and one or more additional amino acids. In some embodiments, the substitution comprises a natural amino acid or a non-natural amino acid. In some embodiments, the variant sequence is a retro inverso amino acid sequence. In some embodiments, a variant sequence comprises one or more additional amino acid residues (e.g., one, two, or three additions) to the N or C terminus. In some embodiments, a variant sequence comprises one or more deletions (e.g., one, two, or three deletions) to amino acid residues at the N or C terminus.

In some embodiments, a variant sequence comprises one or two conservative amino acid substitutions. In some embodiments, the variant sequence further comprises a deletion of one or two terminal amino acids In some embodiments. In some embodiments, a variant sequence comprises one conservative amino acid substitution. In some embodiments, the variant sequence further comprises a deletion of one terminal amino acid. In some embodiments, a variant sequence comprises two conservative amino acid substitutions. In some embodiments, the variant sequence further comprises a deletion of two terminal amino acids. In certain embodiments, the variant sequence inhibits, reduces, or prevents cell death of cells treated with FasL.

Functionality of variant sequences of the peptide (e.g., a variant sequence of the amino acid sequence HHIYLGAVNYIY) can be determined by an in vitro assay. For example, in some embodiments, the variant sequence competes for binding to a Fas receptor (FasR) with Fas ligand (FasL). In some embodiments, the variant sequence inhibits, reduces, or prevents caspase 8 activation in cells treated with FasL (e.g., as measured by commercially available luminescent tetrapeptide cleavage assay kit (Promega, Madison, WI)). In some embodiments, the variant sequence inhibits, reduces, or prevents cell death of cells treated with FasL. By way of further example, in some embodiments, the variant sequence competes for binding to a Fas receptor (FasR) with a Fas-activating antibody (e.g., Fas-agonistic Jo2 monoclonal antibody (BD Biosciences, San Jose, CA)). In some embodiments, the variant sequence inhibits, reduces, or prevents caspase 8 activation in cells treated with a Fas-activating antibody (e.g., as measured by commercially available luminescent tetrapeptide cleavage assay kit (Promega, Madison, WI)). In some embodiments, the variant sequence inhibits, reduces, or prevents cell death of cells treated with a Fas-activating antibody. Accordingly, in some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with Fas ligand (FasL). In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents caspase 8 activation in cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents cell death of cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents caspase 8 activation in cells treated with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents cell death of cells treated with a Fas-activating antibody.

The peptide or a variant sequence thereof can further comprise one or more modifications. In some embodiments, the peptide (e.g., a comprising the amino acid sequence HHIYLGAVNYIY or a variant sequence thereof) comprises a modification. In some embodiments, the peptide is a modified peptide. As used herein, a modification or a modified peptide includes and/or refers to a modification of one or more amino acids in the peptide. In some embodiments, modifications species of stereoisomers. All stereoisomers of the above compounds are contemplated, either in admixture or in pure or substantially pure form. The compounds can have asymmetric centers at any of the atoms. Consequently, the peptide compounds or components thereof can exist in enantiomeric or diastereomeric forms or in mixtures thereof. The present invention contemplates the use of any racemates (i.e., mixtures containing equal amounts of each enantiomer), enantiomerically enriched mixtures (i.e., mixtures enriched for one enantiomer), pure enantiomers or diastereomers, or any mixtures thereof. The chiral centers can be designated as R or S or R, S or d, D, 1, L or d, 1, D, or L. Compounds comprising amino acid residues include residues of D-amino acids, L-amino acids, or racemic derivatives of amino acids. Compounds comprising sugar residues include residues of D-sugars, L-sugars, or racemic derivatives of sugars. Non-limiting examples of modifications are phosphorylation, glycosylation, ubiquitination, nitrosylation, methylation, acetylation, amidation, or lipidation. Modification can be introduced at the C-terminus of the peptide, the N-terminus of the peptide, or at any place in-between. Thus, a modification or a modified peptide includes and/or refers to modifications of the free amino- and/or carboxyl-terminal (N-terminus and C-terminus, respectively). In some embodiments, N-terminal modifications include but are not limited to acetylation, formylation, pyroglutamylation, carbamide addition, lipidation, sulfonamidation, and alkylamination. In some embodiments, C-terminal modifications include but are not limited to amidation, esterification, and incorporation of an aldehyde group. In some embodiments, the modification comprises amidation. In some embodiments, the amidation is at the c-terminus. In some embodiments, the modification comprises a retro inverso peptide (e.g., YIYNVAGLYIHH). In some embodiments, the modification altering the chirality of one or more amino acid residues of the peptide (e.g., L amino acid to D amino acid).

Accordingly, in an embodiment, provided herein are peptides comprising the sequence (a)-HHIYLGAVNYIY-(b) (SEQ ID NO: 1) or (a)-YIYNVAGLYIHH-(b) (SEQ ID NO: 2), or a variant sequence thereof, wherein:
(a) is —H, —OH, —NH₂, G¹(CH₂)ₙ—, R¹CONH—, or R²O—;
(b) is —H, —CH₂OH, —CH₂OR², —CHO, —CO₂R², —CONH₂, —CONHR², —CON(R³)₂, —CONH(CH₂)_{y}NR(³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂, NH-Glu-His-OH, NH-Glu-His-NH₂, -Ala-His-NH₂, -Gly-His-NH₂, —NH-Glu-His-OH, —NH-Glu-His-NH₂, -Ala-His-NH₂, -Gly-His-NH₂, —NH-[D]Glu-[D]-His-OH, —NH-[D]Glu-[D]-His-NH₂, -[D]Ala-[D]-His-NH₂, -Gly[D]-His-NH₂, or —CONH(CH₂)ₙ-G²; G¹, at each occurrence, is independently —H, —C(═O)NH₂, —C(═O)NHR², —C(═O)N(R³)₂, C(═O)OR², or —C(═O)R¹; G² at each occurrence is a heterocyclic ring of 4-7 members comprising at least one tertiary amine functionality NR² within the ring, or a carbocyclic ring of 3-7 members substituted with —N(R³)₂;
R¹, at each occurrence, is independently H, C₁₋₆alkyl, —(CH₂)ₓ(OCH₂CH₂)ₘOR⁵, C₁₋₆-alkoxy or L;
R², at each occurrence, is independently C₁₋₆alkyl, C₂₋₆alkyl substituted with OR⁵ or NR⁵², —(CH₂)ₓ(OCH₂CH₂)ₘOR⁵ or L;
L, at each occurrence, is a multivalent polyethylene glycol derivative with 2-4 termini, each of which may be independently capped with H, R⁵;
R³, at each occurrence, is independently C₁₋₆alkyl, C₂₋₆alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆alkyl, C₁₋₆acyl, or —OPO₃(R⁵)₂;
R⁵, at each occurrence, is independently H or C₁₋₆alkyl;
R⁶, at each occurrence, is H, C₁₋₆alkyl, C₂₋₆hydroxyalkyl, C₁₋₆alkoxy-, C₁₋₆alkyl, or C₁₋₆acyl;

$$m = 1 - 100 ;$$

$$n = 0 - 3 ;$$

$$x = 0 - 6 ; and$$

$$y = 2 - 4 ,$$

wherein at most one of R¹ and R² is L.

In certain instances, provided herein are peptides comprising the structure of Formula I or Formula II, or a pharmaceutically acceptable salt thereof.

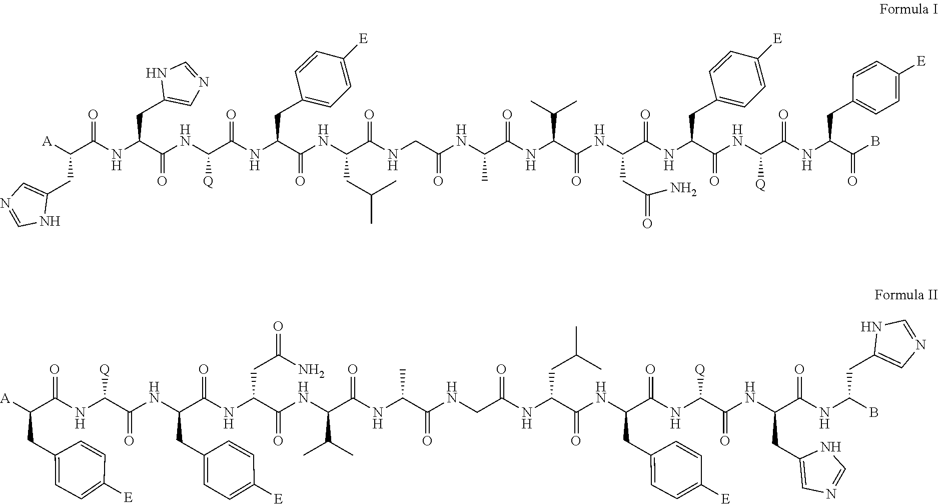

wherein:
A is H—, —OH, —NH₂, G¹(CH₂)ₙ—, R¹CONH—, or R²O—;
B is —H, CH₂OH, CH₂OR², —CHO, —CO₂R², —CONH₂, —CONHR², —CON(R³)₂, —CONH(CH₂)_{y}N(R³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂ NH-Glu-His-OH, NH-Glu-His-NH₂, -Ala-His-NH₂, -Gly-His-NH₂, NH-Glu-His-OH, NH-Glu-His-NH₂, -Ala-His-NH₂, -Gly-His-NH₂, NH-[D]Glu-[D]-His-OH, NH-[D]Glu-[D]-His-NH², -[D]Ala-[D]-His-NH₂, -Gly-[D]-His-NH₂, or CONH(CH₂)ₙ-G²;
E, at each occurrence, is independently —H, —OH, —OR⁴, SH, —SR⁴, or halogen;
G¹, at each occurrence, is independently —H, —C(═O)NH₂, —C(═O)NHR², —C(═O)N(R³)₂, C(═O)OR², or —C(═O)R¹;
G² at each occurrence is a heteroalicyclic ring of 4-7 members comprising at least one tertiary amine functionality NR² within the ring, or a carbocyclic ring of 3-7 members substituted with N(R³)₂;
Q, at each occurrence, is independently, 1-propyl, 2-propyl, 2-methyl-prop-2-yl, C₃₋₆-cycloalkyl, C₄₋₆-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran-4-yltetrahydrothiopyran-2-yl, tetrahydrothiopyran-3-yl, tetrahydrothiopyran-4-yl or 1-CH(OR⁵)CH₃;
R¹, at each occurrence, is independently H, C₁₋₆alkyl, —(CH₂)ₓ(OCH₂CH₂)ₘOR⁵, C₁₋₆alkoxy or L;
R², at each occurrence, is independently C₁₋₆alkyl, C₂₋₆alkyl substituted with OR⁵ or N(R⁵)₂, —(CH₂)ₓ(OCH₂CH₂)ₘOR⁵ or L;
L, at each occurrence, is a multivalent polyethylene glycol derivative with 2-4 termini, each of which may be independently capped with H, R⁵ or another molecule of the peptide of Formula I or II;
R³, at each occurrence, is independently C₁₋₆-alkyl, C₂₋₆-alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆alkyl, C₁₋₆acyl, or —OPO₃(R⁵)₂;
R⁵, at each occurrence, is independently H or C₁₋₆alkyl;
R⁶, at each occurrence, is H, C₁₋₆alkyl, C₂₋₆hydroxyalkyl, C₁₋₆alkoxy-, C₁₋₆alkyl, or C₁₋₆acyl;

$$m = 1 - 100 ;$$

$$n = 0 - 3 ;$$

$$x = 0 - 6 ; and$$

$$y = 2 - 4 ,$$

wherein at most one of R¹ and R² is L.

In some embodiments, provided is a peptide having the structure of Formula III or a pharmaceutically acceptable salt thereof:

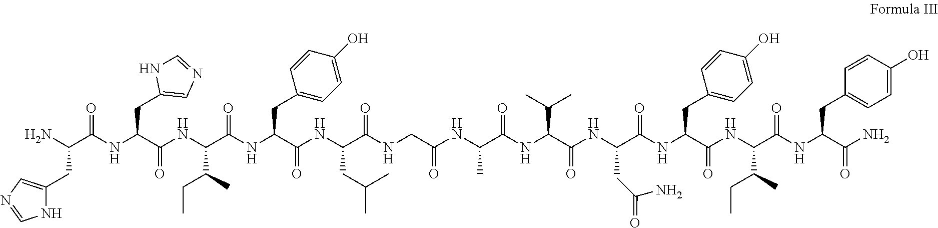 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

In some embodiments, provided is a peptide having the structure of Formula IV or a pharmaceutically acceptable salt thereof:

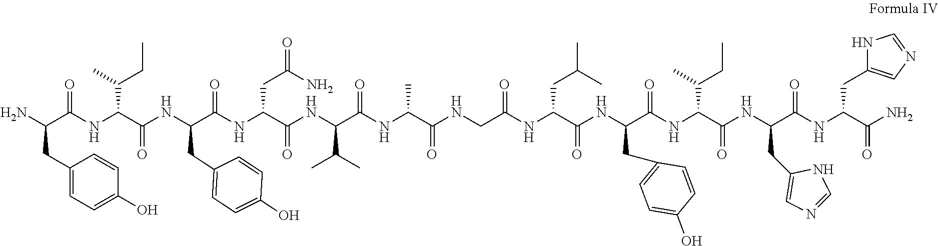 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

In some embodiments, provided is a peptide having the structure of Formula V or a pharmaceutically acceptable salt thereof:

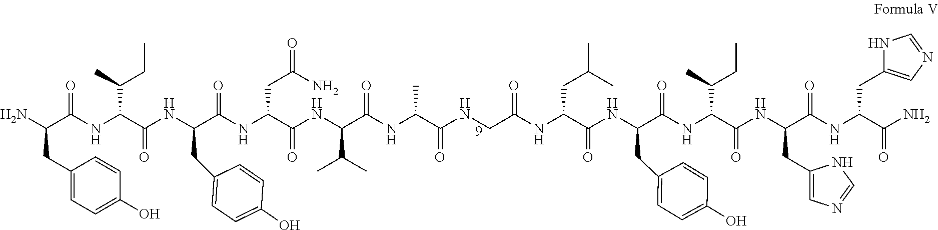 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

In some embodiments, provided is a peptide having the structure of Formula VI or a pharmaceutically acceptable salt thereof:

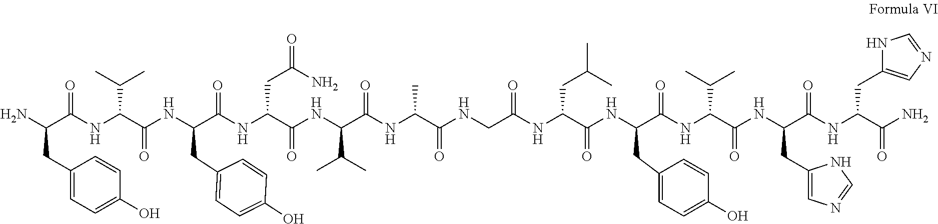 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

In some embodiments, provided is a peptide having the structure of Formula VII or a pharmaceutically acceptable salt thereof:

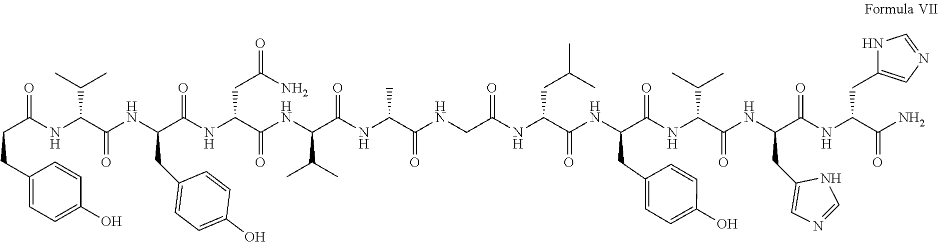

In some embodiments, provided is a peptide having the structure of Formula VIII or a pharmaceutically acceptable salt thereof:

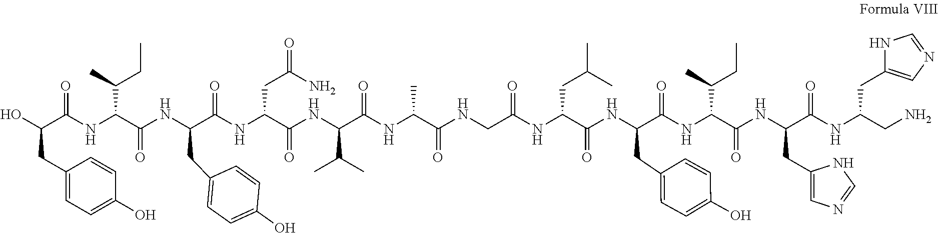 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@@H](CN)Cc1cnc[nH]1)[C@@H](C)CC)C(C)C]

In some embodiments, provided is a peptide having the structure of Formula IX or a pharmaceutically acceptable salt thereof:

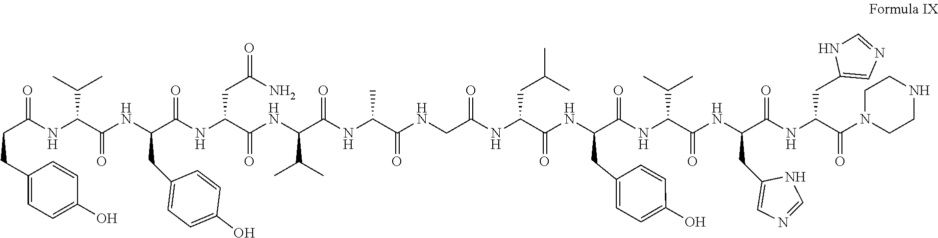

### Salts of Fas Inhibiting Peptides

Further provided herein are salts of the peptide for inhibiting Fas-mediated inflammation in the eye and for use in the methods described here. As used herein, salt is generally synonymous with pharmaceutically acceptable salts, and/or includes or refers to pharmaceutically acceptable salts. Examples of pharmaceutically acceptable salts are salts with organic or inorganic acids such as (but not limited to) include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared using methods well known in the art. In some embodiments, the salt is a hydrochloride salt.

In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, and polyamine resins.

Salts and pharmaceutically acceptable salts are described in in J. Pharmaceutical Sciences, 66: 1-19 (1977), the contents of which are incorporated by reference herein.

In some embodiments, the salt is an acetate salt. In some embodiments, the acetate salt is a poly-acetate salt. In some embodiments, the poly-acetate salt is a tri-acetate salt.

### Pharmaceutical Compositions

In an embodiment, further provided are pharmaceutical compositions (also referred to as compositions) comprising the Fas inhibiting peptides. In some embodiments, the pharmaceutical compositions described herein comprise the peptide (e.g., a peptide comprising the amino acid sequence HHIYLGAVNYIY) or a pharmaceutically acceptable salt thereof. In some embodiments, the pharmaceutical compositions described herein comprise the peptide comprising the amino acid sequence HHIYLGAVNYIY or variant sequence thereof, or a pharmaceutically acceptable salt of the peptide. In some embodiments, the pharmaceutical compositions described herein comprise the peptide having the structure of any one of Formulas I-IX or a pharmaceutically acceptable salt thereof. In some embodiments, the pharmaceutical compositions described herein comprise the peptide having the structure of Formula I or a pharmaceutically acceptable salt thereof. In some embodiments, the pharmaceutical compositions described herein comprise the peptide having the structure of Formula III or a pharmaceutically acceptable salt thereof.

The pharmaceutical composition can comprise one or more excipients. As used herein, an excipient includes and/or refers to any pharmaceutically acceptable additive, carrier, diluent, adjuvant, or other ingredient, other than the active pharmaceutical ingredient (API), which is typically included for formulation and/or administration to a patient. A pharmaceutical composition can comprise a single pharmaceutical formulation (e.g., extended release, immediate release, delayed release, nanoparticulate, etc.) or multiple formulations (e.g., immediate release and delayed release, nanoparticulate and nonnanoparticulate, etc.). An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

In some embodiments, the compositions comprise excipients that are suitable for ocular application. Suitable excipients and include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1, and the like. Suitable chelating agents include sodium edetate and the like. Suitable buffering agents include phosphates, borates, citrates, acetates, tromethamine, and the like. Suitable surfactants include ionic and nonionic surfactants. In some embodiments, the one or more excipients comprises nonionic surfactants, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

In some embodiments, the composition comprises a non-ionic surfactant. In some embodiments, the composition comprises a polysorbate, a polyethoxylated castor oil derivative, a polyethoxylated fatty acid, a polyethoxylated alcohol, a polyoxyethylene-polyoxypropylene block copolymer (Poloxamer), or an oxyethylated tertiary octylphenol formaldehyde polymer (Tyloxapol). In some embodiments, the composition comprises a polysorbate. In some embodiments, the composition comprises a polyethoxylated castor oil derivative. In some embodiments, the composition comprises a polyethoxylated fatty acid. In some embodiments, the composition comprises a polyethoxylated alcohol. In some embodiments, the composition comprises a polyoxyethylene-polyoxypropylene block copolymer (Poloxamer). In some embodiments, the composition comprises an oxyethylated tertiary octylphenol formaldehyde polymer (Tyloxapol). In some embodiments, the surfactant makes up 0.05%-20% weight per weight (w/w) of the composition. In some embodiments, the non-ionic surfactant is about 0.05% w/w of the composition to about 20% w/w of the composition. In some embodiments, the non-ionic surfactant is at least about 0.05% w/w of the composition. In some embodiments, the non-ionic surfactant is at most about 20% w/w of the composition. In some embodiments, the non-ionic surfactant is about 0.05% w/w of the composition to about 0.1% w/w of the composition, about 0.05% w/w of the composition to about 0.5% w/w of the composition, about 0.05% w/w of the composition to about 1% w/w of the composition, about 0.05% w/w of the composition to about 2% w/w of the composition, about 0.05% w/w of the composition to about 5% w/w of the composition, about 0.05% w/w of the composition to about 10% w/w of the composition, about 0.05% w/w of the composition to about 20% w/w of the composition, about 0.1% w/w of the composition to about 0.5% w/w of the composition, about 0.1% w/w of the composition to about 1% w/w of the composition, about 0.1% w/w of the composition to about 2% w/w of the composition, about 0.1% w/w of the composition to about 5% w/w of the composition, about 0.1% w/w of the composition to about 10% w/w of the composition, about 0.1% w/w of the composition to about 20% w/w of the composition, about 0.5% w/w of the composition to about 1% w/w of the composition, about 0.5% w/w of the composition to about 2% w/w of the composition, about 0.5% w/w of the composition to about 5% w/w of the composition, about 0.5% w/w of the composition to about 10% w/w of the composition, about 0.5% w/w of the composition to about 20% w/w of the composition, about 1% w/w of the composition to about 2% w/w of the composition, about 1% w/w of the composition to about 5% w/w of the composition, about 1% w/w of the composition to about 10% w/w of the composition, about 1% w/w of the composition to about 20% w/w of the composition, about 2% w/w of the composition to about 5% w/w of the composition, about 2% w/w of the composition to about 10% w/w of the composition, about 2% w/w of the composition to about 20% w/w of the composition, about 5% w/w of the composition to about 10% w/w of the composition, about 5% w/w of the composition to about 20% w/w of the composition, or about 10% w/w of the composition to about 20% w/w of the composition. In some embodiments, the non-ionic surfactant is about 0.05% w/w of the composition, about 0.1% w/w of the composition, about 0.5% w/w of the composition, about 1% w/w of the composition, about 2% w/w of the composition, about 5% w/w of the composition, about 10% w/w of the composition, or about 20% w/w of the composition.

In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the non-ionic surfactant comprises Polysorbate 20, Poloxamer 407, Tyloxapol, or cremophor. In some embodiments, the non-ionic surfactant is Polysorbate 20. In some embodiments, the non-ionic surfactant is Poloxamer 407. In some embodiments, the non-ionic surfactant is Tyloxapol. In some embodiments, the non-ionic surfactant is cremophor. The non-ionic surfactants described herein can be present within any one of the ranges (e.g., percent w/w) described herein, a specific value that falls within the described ranges.

In some embodiments, the composition further comprises cosolvents (e.g., between 0.5 and 50% w/w), such as N,N-Dimethylacetamide, ethanol, PEG-400, propylene glycol, dimethylsulfoxide (DMSO); oils, or cyclodextrins may be added to a pharmaceutical preparation. In some embodiments, the composition further comprises a tonicity-adjusting agent. In some embodiments, the composition is an isotonic solution. In some embodiments, the tonicity-adjusting agent is mannitol, sorbitol, glucose or trehalose, or an inorganic salt such as sodium chloride. In some embodiments, the composition comprises mannitol. In some embodiments, the composition comprises sorbitol. In some embodiments, the composition comprises glucose or trehalose. In some embodiments, the composition comprises an inorganic salt. In some embodiments, the tonicity-adjusting agent is present at an amount suitable to bring the tonicity of the composition into the 250-400 mOsm/L range. In some embodiments, the non-ionic surfactant is about 1% w/w of the composition to about 10% w/w of the composition. In some embodiments, the non-ionic surfactant is at least about 1% w/w of the composition. In some embodiments, the non-ionic surfactant is at most about 10% w/w of the composition. In some embodiments, the non-ionic surfactant is about 1% w/w of the composition to about 2% w/w of the composition, about 1% w/w of the composition to about 3% w/w of the composition, about 1% w/w of the composition to about 4% w/w of the composition, about 1% w/w of the composition to about 5% w/w of the composition, about 1% w/w of the composition to about 10% w/w of the composition, about 2% w/w of the composition to about 3% w/w of the composition, about 2% w/w of the composition to about 4% w/w of the composition, about 2% w/w of the composition to about 5% w/w of the composition, about 2% w/w of the composition to about 10% w/w of the composition, about 3% w/w of the composition to about 4% w/w of the composition, about 3% w/w of the composition to about 5% w/w of the composition, about 3% w/w of the composition to about 10% w/w of the composition, about 4% w/w of the composition to about 5% w/w of the composition, about 4% w/w of the composition to about 10% w/w of the composition, or about 5% w/w of the composition to about 10% w/w of the composition. In some embodiments, the non-ionic surfactant is about 1% w/w of the composition, about 2% w/w of the composition, about 3% w/w of the composition, about 4% w/w of the composition, about 5% w/w of the composition, or about 10% w/w of the composition.

In some embodiments, the composition comprises a buffering agent. In some embodiments, the buffering agent is an acidifying agent. In some embodiments, the acidifying agent is an acetate buffer at pH 4.5. In some embodiments, the concentration acetate buffer pH 4.5 is about 10 millimolar (mM). Generally, the pH may be controlled by an appropriate buffer suitable for injection into the eye, for example the pH of the composition can be in the 3.0-7.5 range or 3.5-4.5 range.

As described herein, the composition can comprise one or more excipients. Accordingly, in some embodiments, the composition comprises a non-ionic surfactant, a tonicity-adjusting agent, and a buffering agent, in combination with the peptide. Any of the described excipients can be combined within the amounts and/or ranges described.

### Dosages and Dosing Regimens

In an embodiment, the compositions described herein comprise an amount of the peptide suitable to inhibit Fas-mediated inflammation and/or treat, inhibit, reduce, and/or prevent retinal degeneration or the symptoms thereof in an eye. As used herein, a dose or dosage includes and/or refers to the amount of therapeutic agent, such as the peptides described, in a composition (e.g., a composition for administering to an eye). A dose can refer to either (i) the peptide (parent compound) or the pharmaceutically acceptable salt thereof. In some embodiments, the amount of the peptide in the composition (i.e., pharmaceutical composition) that is suitable for the methods described herein (e.g., treating retinal degeneration) ranges from 5 micrograms (ug) to 10,000 ug. The dosing forms comprising the compositions described herein are generally administered to the vitreous humor of an eye and can be further formulated for injection into the eye (e.g., intravitreal injection). In some embodiments, the amount of the peptide (e.g., a peptide comprising the amino acid sequence HHIYLGAVNYIY) or a pharmaceutically acceptable salt thereof that is suitable for the methods described herein ranges from 5 ug to 10,000 ug. In some embodiments, the amount of the peptide comprising the amino acid sequence HHIYLGAVNYIY or variant sequence thereof, or a pharmaceutically acceptable salt of the peptide that is suitable for the methods described herein ranges from 5 ug to 10,000 ug. In some embodiments, the amount of the peptide having the structure of Formula I or a pharmaceutically acceptable salt thereof that is suitable for the methods described herein ranges from 5 ug to 10,000 ug. In some embodiments, the amount of the peptide having the structure of Formula III or a pharmaceutically acceptable salt thereof that is suitable for the methods described herein ranges from 5 ug to 10,000 ug.

In some embodiments, a dose comprises about 5-1,000 ug of the peptide (e.g., Formula III) or the variant sequence thereof. In some embodiments, a dose comprises about 25-500 ug of the peptide or the variant sequence thereof. In some embodiments, a dose comprises about 25-250 ug of the peptide or the variant sequence thereof. In some embodiments, a dose comprises about 50-250 ug of the peptide or the variant sequence thereof. In certain embodiments, a dose comprises about 50 ug of the peptide or the variant sequence thereof. In certain embodiments, a dose comprises about 100 ug of the peptide or the variant sequence thereof. In certain embodiments, a dose comprises about 200 ug of the peptide or the variant sequence thereof. In certain embodiments, a dose comprises about 300 ug of the peptide or the variant sequence thereof. In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

In some embodiments, a dose comprises about 5 ug of a pharmaceutically acceptable salt of the peptide to about 300 ug of a pharmaceutically acceptable salt of the peptide. In some embodiments, a dose comprises at least about 5 ug of a pharmaceutically acceptable salt of the peptide. In some embodiments, a dose comprises at most about 300 ug of a pharmaceutically acceptable salt of the peptide.

The concentration of the peptide within the composition can be adjusted in a manner suitable for ocular administration. In some embodiments, the concentration of the peptide within the composition ranges from about 0.1 milligrams per milliliter (mg/mL) to about 5 mg/mL. In some embodiments, the concentration of the peptide within the composition ranges from about 0.1 milligrams per milliliter (mg/mL) to about 10 mg/mL. In some embodiments, the concentration of the peptide within the composition ranges from about 0.1 milligrams per milliliter (mg/mL) to about 100 mg/mL. In some embodiments, the concentration of the peptide (e.g., a peptide comprising the amino acid sequence HHIYLGAVNYIY) or a pharmaceutically acceptable salt thereof ranges from about 0.1 mg/mL to about 5 mg/mL. In some embodiments, the concentration of the peptide comprising the amino acid sequence HHIYLGAVNYIY or variant sequence thereof, or a pharmaceutically acceptable salt of the peptide ranges from about 0.1 milligrams per milliliter (mg/mL) to about 5 mg/mL. In some embodiments, the concentration of the peptide having the structure of Formula I or a pharmaceutically acceptable salt thereof ranges from about 0.1 mg/mL to about 5 mg/mL. In some embodiments, the concentration of the peptide having the structure of Formula III or a pharmaceutically acceptable salt thereof ranges from about 0.1 mg/mL to about 5 mg/mL.

### Pharmacokinetics

In some embodiments, the compositions described herein are administered to an eye of an individual in need thereof. Administration to an eye (i.e., “ocular application” or “ocular administration”) includes subconjunctival, intravitreal, retrobulbar, intracameral administration subretinal, or suprachoroidal. In some embodiments, ocular administration comprises subconjunctival, intravitreal, retrobulbar, or intracameral administration. In some embodiments, ocular administration comprises intravitreal administration. In some embodiments, ocular administration comprises subconjunctival administration. In some embodiments, ocular administration comprises retrobulbar administration. In some embodiments, ocular administration comprises intracameral administration.

In some embodiments, the dosing forms comprising the compositions described herein are generally administered to the vitreous humor of an eye. In some embodiments, the half-life of the peptide (e.g., a peptide comprising the amino acid sequence HHIYLGAVNYIY) or a pharmaceutically acceptable salt thereof in the vitreous humor is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 30 days to greater than about 275 days. In some embodiments, the peptide having the structure of Formula I or a pharmaceutically acceptable salt thereof has a half-life in the vitreous humor that is greater than is greater than about 30 days to greater than about 275 days. In some embodiments, the peptide having the structure of Formula III or a pharmaceutically acceptable salt thereof has a half-life in the vitreous humor that is greater than is greater than about 30 days to greater than about 275 days. In some embodiments, the peptide comprising the amino acid sequence HHIYLGAVNYIY or variant sequence thereof, or a pharmaceutically acceptable salt of the peptide has a half-life in the vitreous humor that is greater than about 14 days to greater than about 275 days. In some embodiments, the peptide having the structure of Formula I or a pharmaceutically acceptable salt thereof has a half-life in the vitreous humor that is greater than is greater than about 14 days to greater than about 275 days. In some embodiments, the peptide having the structure of Formula III or a pharmaceutically acceptable salt thereof has a half-life in the vitreous humor that is greater than is greater than about 14 days to greater than about 275 days.

In some embodiments, the half-life of the peptide is greater than about 14 days in the eye. In some embodiments, the half-life of the peptide is greater than about 30 days in the eye. In some embodiments, the half-life of the peptide is greater than about 60 days in the eye. In some embodiments, the half-life of the peptide is greater than about 90 days in the eye. In some embodiments, the half-life of the peptide is greater than about 120 days in the eye. In some embodiments, the half-life of the peptide is greater than about 150 days in the eye. In some embodiments, the half-life of the peptide is greater than about 180 days in the eye. In some embodiments, the half-life of the peptide is greater than about 210 days in the eye. In some embodiments, the half-life of the peptide is greater than about 240 days in the eye. In some embodiments, the half-life of the peptide is greater than about 270 days in the eye.

In some embodiments, the half-life of the peptide is greater than about 14 days in the vitreous humor. In some embodiments, the half-life of the peptide is greater than about 30 days in the vitreous humor. In some embodiments, the half-life of the peptide is greater than about 60 days in the vitreous humor. In some embodiments, the half-life of the peptide is greater than about 90 days in the vitreous humor. In some embodiments, the half-life of the peptide is greater than about 120 days in the vitreous humor. In some embodiments, the half-life of the peptide is greater than about 150 days in the vitreous humor. In some embodiments, the half-life of the peptide is greater than about 180 days in the vitreous humor. In some embodiments, the half-life of the peptide is greater than about 210 days in the vitreous humor. In some embodiments, the half-life of the peptide is greater than about 240 days in the vitreous humor. In some embodiments, the half-life of the peptide is greater than about 270 days in the vitreous humor.

Determining the amount of the peptide in the vitreous humor generally requires collecting all of the vitreous fluid or a substantial portion thereof from an eye or sacrificing the eye in order to sample the vitreous humor. In some embodiments, collecting all of the vitreous fluid or a substantial portion thereof in a human eye, or sacrificing an eye is not feasible for maintaining the health of an eye in a human. Accordingly, in some embodiments, the half-life of the peptide in a human eye is determined by measuring and/or extrapolating from a half-life of the peptide in the eye of a mammal. In some embodiments, the mammal is a rabbit. In some embodiments, the mammal is a pig (e.g., minipig). In some embodiments, the mammal is a monkey. Various methods of detecting the presence of a drug are also suitable for detecting the peptide. For example, methods suitable for detecting the peptide include performing mass spectrometry (e.g., liquid chromatography-mass spectrometry (LC-MS) or high-performance LC-MS (HPLC-MS)) on a sample from the vitreous humor.

### Methods

Provided herein are methods that are advantageous for use in improving visual function in an individual having an ocular disease and/or disorder, wherein the methods utilize multiple doses of a Fas inhibitor. In some embodiments, the ocular disease and/or disorder comprises a loss in visual function (e.g., best corrected visual acuity, field of vision or visual field, contrast sensitivity, binocular function, low luminance acuity, low contrast acuity, color vision, perimetry, threshold sensitivity, reading speed, and/or light/dark adaptation). In some embodiments, the ocular disease and/or disorder comprises photoreceptor cell death. In some embodiments, the ocular disease and/or disorder comprises retinal pigment epithelial cell death. In some embodiments, the ocular disease and/or disorder comprises degeneration of the macula. In some embodiments, the ocular disease and/or disorder comprises inflammation within the eye and/or retinal tissue (e.g., as measured by pro-inflammatory markers within a sample from the vitreous). In some embodiments, the ocular disease and/or disorder comprises retinal ganglion cell death.

Provided herein are methods of improving visual function in an eye of an individual having an ocular disease or disorder, the method comprising: (a) administering a first composition comprising Fas inhibitor; and (b) administering a second composition comprising the Fas inhibitor, wherein the second composition is administered about 10 weeks or more after administering the first composition. Also provided herein are methods of improving visual function (e.g., BCVA or visual field) in an eye of an individual having an ocular disease or disorder, comprising: administering a plurality of compositions to the eye, wherein: each composition of the plurality of compositions comprises a Fas inhibitor; and no greater than five compositions are administered to the eye within a year. In some embodiments, the Fas inhibitor is a peptide, a polypeptide (e.g., FasR or FasL), an antibody that binds FasR, or antibody that binds FasL.

Provided herein are methods of improving visual function (e.g., BCVA or visual field) in an eye of an individual having an ocular disease or disorder, comprising: (a) administering a first composition comprising a peptide to the eye of the individual, the peptide comprising an amino acid sequence HHIYLGAVNYIY or a variant sequence thereof, or a pharmaceutically acceptable salt of the peptide; and (b) administering a second composition comprising the peptide to the eye, wherein the second composition is administered about 10 weeks or more after administering the first composition. Also provided are methods of improving visual function (e.g., BCVA or visual field) in an eye of an individual having an ocular disease or disorder, comprising: (a) administering a first composition comprising a peptide to the eye of the individual, the peptide having the structure of Formula I or a pharmaceutically acceptable salt thereof; and (b) administering a second composition comprising the peptide to the eye, wherein the second composition is administered about 10 weeks or more after administering the first composition. Further provided are methods of improving visual function (e.g., BCVA or visual field) in an eye of an individual having an ocular disease or disorder, comprising: (a) administering a first composition comprising a peptide to the eye of the individual, the peptide having the structure of Formula III or a pharmaceutically acceptable salt thereof; and (b) administering a second composition comprising the peptide to the eye, wherein the second composition is administered about 10 weeks or more after administering the first composition.

In some embodiments, the second composition is administered about 8 weeks or more after administering the first composition. In some embodiments, the second composition is administered about 10 weeks or more after administering the first composition. In some embodiments, the second composition is administered about 12 weeks or more after administering the first composition. In some embodiments, the second composition is administered about 16 weeks or more after administering the first composition. In some embodiments, the second composition is administered about 20 weeks or more after administering the first composition. In some embodiments, the second composition is administered about 24 weeks or more after administering the first composition.

In some embodiments, the visual function is improved after administering the first composition, wherein administering the second composition maintains the improvement in visual function. In some embodiments, the visual function is improved after administering the second composition. In some embodiments, the visual function is improved after administering the first composition and is further improved after administering the second composition.

Further provided herein are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide comprising an amino acid sequence HHIYLGAVNYIY or variant sequence thereof, or a pharmaceutically acceptable salt thereof; and no greater than five compositions are administered to the eye within a year. Also provided are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide having the structure of Formula I or a pharmaceutically acceptable salt thereof; and no greater than five compositions are administered to the eye within a year. Additionally provided are methods of improving visual function (e.g., BCVA or visual field) in an eye of an individual having an ocular disease or disorder, comprising: administering a plurality of compositions to the eye, wherein: each composition of the plurality of compositions comprises a peptide, the peptide having the structure of Formula III or a pharmaceutically acceptable salt thereof; and no greater than five compositions are administered to the eye within a year.

In some embodiments, no greater than four compositions are administered to the eye within a year. In some embodiments, no greater than three compositions are administered to the eye within a year. In some embodiments, no greater than two compositions are administered to the eye within a year. In some embodiments, the plurality of compositions comprises 2 compositions. In some embodiments, the plurality of compositions comprises 3 compositions. In some embodiments, the plurality of compositions comprises 4 compositions. In some embodiments, the plurality of compositions comprises 5 compositions.

In some embodiments, the visual function is improved after administering a first composition, wherein administering subsequence compositions maintain the improvement in visual function. In some embodiments, the visual function is improved after administering multiple compositions (e.g., two or more). In some embodiments, the visual function is improved after administering a first composition and is further improved after administering an additional one (e.g., a second) or more (e.g., a third or fourth) composition.

In some embodiments, the method comprises administering each composition to the vitreous humor of the eye. In some embodiments, the peptide has a half-life in the vitreous humor greater than about 30 days. In some embodiments, the peptide has a half-life in the vitreous humor greater than about 90 days. In some embodiments, the peptide has a half-life in the vitreous humor greater than about 180 days. In some embodiments, the peptide has a half-life in the vitreous humor greater than about 200 days. In some embodiments, the method comprises using the vitreous humor as a depot to provide the peptide to retinal tissue in the eye. In certain embodiments, the peptide is present in the vitreous humor greater than about 8 weeks after administration. In certain embodiments, the peptide is present in the vitreous humor greater than about 10 weeks after administration. In certain embodiments, the peptide is present in the vitreous humor greater than about 12 weeks after administration. In certain embodiments, the peptide is present in the vitreous humor greater than about 16 weeks after administration. In certain embodiments, the peptide is present in the vitreous humor greater than about 20 weeks after administration. In certain embodiments, the peptide is present in the vitreous humor greater than about 24 weeks after administration. In certain embodiments, the peptide is present in the vitreous humor greater than about 30 weeks after administration. In certain embodiments, the peptide is present in the vitreous humor greater than about 40 weeks after administration. In certain embodiments, the peptide is present in the vitreous humor greater than about 50 weeks after administration.

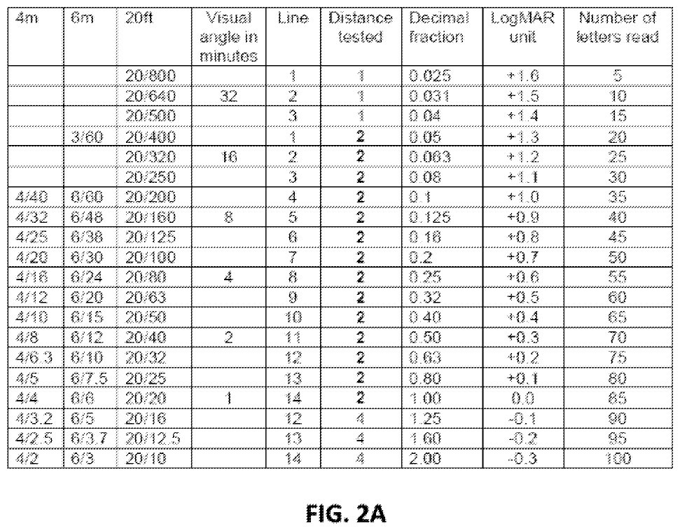

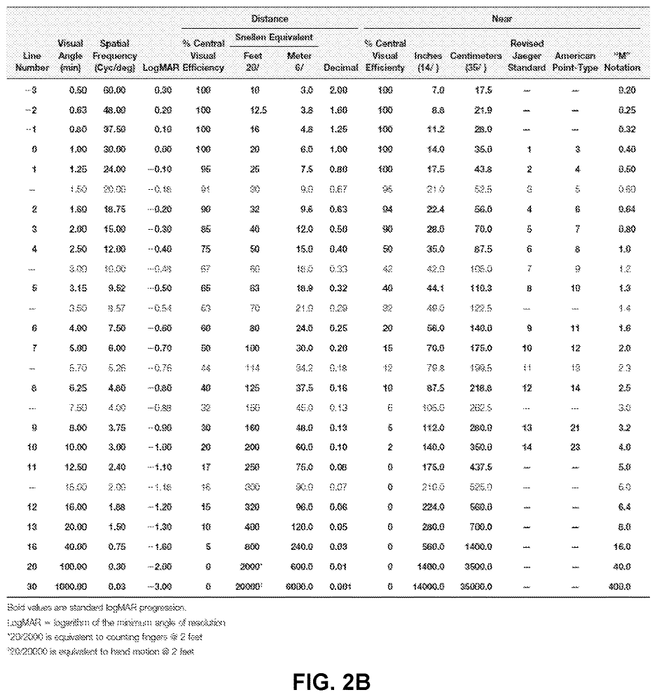

In some embodiments, improving or treating visual function comprises improving visual function as compared to a baseline visual function prior to administering the peptide or the variant sequence thereof, or the pharmaceutically acceptable salt of the peptide. In some embodiments, visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, visual function comprises best corrected visual acuity (e.g., increase in visual acuity from 20/200 to 20/100, or an increase in visual acuity from 20/50 to 20/40, and/or an increase in visual acuity from measured in a reduction in log MAR, etc.). As used herein, the term “best corrected visual acuity” or “BCVA”, generally refers to the minimum angle of resolution subtended by a certain number of arc minutes. In certain instances, the subtended visual angle of an object is the angle formed by rays projecting from the eye to the top and bottom (or left and right sides) of an object. In such instances, such visual angles are used to indicate the size of the retinal image of the object (e.g., the larger the visual angle, the larger the retinal image size is). In certain instances, the visual angle is influenced by two parameters: the size of the object and the distance of the object from the eye. Bigger objects cast larger images on the retina than smaller objects. Thus, the larger the object is, the larger its visual angle will be. Closer objects cast larger images on the retina than smaller objects. Thus, the closer the object is to the eye, the larger its visual angle will be. By way of example, standard vision is thus the ability to distinguish features separated by 1 minute of arc. By way of further example, the ability to distinguish a set of bars separated by 1 arc minute is 20/20 vision, 2 arc minutes is 20/40 vision. In some embodiments, visual acuity is measured by a Snellen chart, ETDRS chart, Landolt C chart, Tumbling E chart, HOTV chart, and/or log MAR (log minimum angle of resolution). FIG. 2A-2B show an exemplary, but non-limiting, conversion chart for comparing BCVA values. In some embodiments, visual function comprises field of vision or visual field (e.g., Humphrey's visual field as measured by an HFA). In some embodiments, visual function comprises contrast sensitivity. In some embodiments, visual function comprises binocular function. In some embodiments, visual function comprises low luminance acuity. In some embodiments, visual function comprises low contrast acuity. In some embodiments, visual function comprises color vision. In some embodiments, visual function comprises perimetry. In some embodiments, visual function comprises threshold sensitivity. In some embodiment, visual function comprises reading speed. In some embodiments, visual function comprises light/dark adaptation. In some embodiments, improving visual function comprises improving visual function as compared to a baseline visual function prior to the administering peptide or the variant sequence thereof, or the pharmaceutically acceptable salt of the peptide.

In some embodiments, the methods described herein include a methods of treating and/or improving vision loss associated with inflammation (e.g., Fas-mediated inflammation) in an eye. In some embodiments, retinal inflammation can be determined by observing the symptoms associated with inflammation in the eye (e.g., loss of and/or decrease in visual acuity, central vision loss, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-1b, IP-10, IL-18, MIP-1a, IL-6, GFAP, MIP2, MCP-1, or MIP-1b); a Fas-mediated complement-related molecules (complement component 3 (C3) or complement component 1 q (C1q)) Caspase 8; components of the inflammasome (e.g., NLRP3 or NLRP2); C—X—C motif chemokines (e.g., CXCL2 (MIP-2alpha) or CXCL10 (IP-10)); C—X3-C motif chemokines (e.g., CX3CL1 (fractalkine)); C—C motif chemokines (CCL2 (MCP-1), CCL3 (MIP-1a), and CCL4 (MIP-1b)); toll-like receptor 4 (TLR4); interleukin cytokines (e.g., IL-1b, IL-18, and IL-6); TNF superfamily cytokines (e.g., TNFa); or GFAP.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions. In some embodiments, the variant sequence comprises a truncation.

In some embodiments, the peptide further comprises a modification. In some embodiments, comprises a modified amino acid or a non-natural amino acid. In some embodiments, the peptide comprises an amidated C-terminus. In some embodiments, the peptide has the structure of Formula I, or a pharmaceutically acceptable salt thereof. In some embodiments, the peptide has the structure of Formula III, or a pharmaceutically acceptable salt thereof.

In some embodiments, the pharmaceutically acceptable salt of the peptide is administered. In some embodiments, the pharmaceutically acceptable salt is an acetate salt. In some embodiments, the pharmaceutically acceptable salt is a polyacetate salt. In some embodiments, the polyacetate salt is a triacetate salt. In some embodiments, the pharmaceutically acceptable salt is a hydrochloride salt.

In some embodiments, the peptide or the variant sequence thereof, or the pharmaceutically acceptable salt of the peptide is formulated in a composition (e.g., the pharmaceutical compositions described herein).

In some embodiments, about 5-1,000 ug of the peptide or the variant sequence thereof, or the pharmaceutically acceptable salt of the peptide is administered. In some embodiments, about 25-500 ug of the peptide or the variant sequence thereof, or the pharmaceutically acceptable salt of the peptide is administered. In some embodiments, about 25-250 ug of the peptide or the variant sequence thereof, or the pharmaceutically acceptable salt of the peptide is administered. In some embodiments, about 50-250 ug of the peptide or the variant sequence thereof, or the pharmaceutically acceptable salt of the peptide is administered. In certain embodiments, about 50 ug of the peptide or the variant sequence thereof, or the pharmaceutically acceptable salt of the peptide is administered. In certain embodiments, about 100 ug of the peptide or the variant sequence thereof, or the pharmaceutically acceptable salt of the peptide is administered. In certain embodiments, about 200 ug of the peptide or the variant sequence thereof, or the pharmaceutically acceptable salt of the peptide is administered. For example, in methods comprising treating glaucoma and improving visual function, about 50 ug or 100 ug of a peptide having the structure of Formula III is administered. For example, in methods comprising treating macular degeneration and improving visual function, about 50 ug or 200 ug of a peptide having the structure of Formula III is administered. In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

In some embodiments, the ocular disease or disorder comprises retinal epithelial cell death. In some embodiments, the ocular disease or disorder comprises retinal ganglion cell death. In some embodiments, the ocular disease or disorder comprises photoreceptor cell death.

In some embodiments, the ocular disease or disorder is macular degeneration. As used herein, macular degeneration generally includes and/or refers to any of a number of conditions in which the retinal macula degenerates and/or becomes dysfunctional (e.g., as a consequence of decreased growth of cells of the macula, increased death or rearrangement of the cells of the macula (e.g., RPE cells), loss of normal biological function, or a combination thereof). Macular degeneration generally, results in the loss of integrity of the histological architecture of the cells and/or extracellular matrix of the normal macula and/or the loss of function of the cells of the macula. As used herein, macular degeneration also includes and/or refers to extramacular changes that occur prior to or following dysfunction and/or degeneration of the macula. In some embodiments, the macular degeneration is age-related macular degeneration. In some embodiments, the macular degeneration comprises geographic atrophy. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors

In some embodiments, the ocular disease or disorder is glaucoma. As used herein, glaucoma generally includes and refers to a group of ocular diseases, which cause progressive damage to the optic nerve and resultant optical field defects, vision loss and, in some cases, blindness. In some embodiments, glaucoma is accompanied by abnormally high intraocular pressure and/or inflammation in the eye (e.g., within the retinal tissue). In some embodiments, glaucoma is not accompanied by abnormally high intraocular pressure and/or inflammation in the eye (e.g., within the retinal tissue. In some embodiments, the ocular disease, disorder, or condition comprises the loss and/or death of retinal ganglion cells.

In some embodiments, the glaucoma is progressing glaucoma. In certain embodiments, progressing glaucoma includes a declining Humphrey Visual Field (HVF) mean deviation (MD). In certain embodiments, progressing glaucoma comprises a Humphrey Visual Field (HVF) mean deviation (MD) decline of −0.50 dB/year or worse (e.g., −1 dB/year). In certain embodiments, progressing glaucoma comprises a Humphrey Visual Field (HVF) mean deviation (MD) decline of −1 dB/year or worse (e.g., −2 dB/year). In certain embodiments, progressing glaucoma comprises a Humphrey Visual Field (HVF) mean deviation (MD) decline of −2 dB/year or worse. In certain embodiments, progressing glaucoma comprises a Humphrey Visual Field (HVF) mean deviation (MD) decline of −3 dB/year or worse. In certain embodiments, progressing glaucoma comprises a Humphrey Visual Field (HVF) mean deviation (MD) decline of −4 dB/year or worse. In certain embodiments, progressing glaucoma comprises a Humphrey Visual Field (HVF) mean deviation (MD) decline of −5 dB/year or worse. For example, the Examples described herein show data for treating progressing glaucoma, including the treatment of a number of different Humphrey Visual Field (HVF) mean deviation (MD) declines. In certain embodiments, progressing glaucoma comprises a Humphrey Visual Field (HVF) mean deviation (MD) decline between about −1 dB/year and about −3 dB/year. In certain embodiments, progressing glaucoma comprises a Humphrey Visual Field (HVF) mean deviation (MD) decline between about −1 dB/year and about −4 dB/year. In certain embodiments, progressing glaucoma comprises a Humphrey Visual Field (HVF) mean deviation (MD) decline between about −1 dB/year and about −5 dB/year.

In certain embodiments, progressing glaucoma comprises a Humphrey Visual Field (HVF) mean deviation (MD) decline between about −0.5 dB/year and about −3 dB/year. In certain embodiments, progressing glaucoma comprises a Humphrey Visual Field (HVF) mean deviation (MD) decline between about −0.5 dB/year and about −4 dB/year. In certain embodiments, progressing glaucoma comprises a Humphrey Visual Field (HVF) mean deviation (MD) decline between about −0.5 dB/year and about −5 dB/year.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, the term “inhibition” or “inhibiting” includes and/or refers to the reduction or suppression of a given condition, symptom, disorder, or disease, and/or a decrease in the baseline activity of a biological activity or process.

As used herein, the term “treating” or “treatment” of includes and/or refers to ameliorating the disease or disorder or symptoms thereof (e.g., slowing or arresting or reducing the development of the disease or at least one of the clinical symptoms thereof). In certain embodiments, “treating” or “treatment” also includes and/or refers to alleviating or ameliorating at least one physical and/or biological parameters including those which may not be discernible by the patient. In certain embodiments, “treating” or “treatment” includes and/or refers to modulating a disease, disorder, or biological process either physically, (e.g., stabilization of a discernible symptom), physiologically, (e.g., stabilization of a physical and/or biological parameter), or both. In certain embodiments, “treating” or “treatment” includes and/or refers to preventing or delaying the onset or development or progression of the disease or disorder. In certain embodiments, “treating” or “treatment” includes and/or refers to preventing or delaying or inhibiting the deterioration of (i) a healthy physiological state or (ii) a baseline physiological state (e.g., the progression of a disease or disorder).

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

### EXAMPLES

### Example 1—Improving Visual Outcomes in Open Angle Glaucoma Patients

### Objective

The primary objective was to determine multi-dose safety of a peptide having the structure of Formula III (ONL1204) and select doses in patients having open-angle glaucoma. The primary endpoints were the safety profile demonstrated in this study, including: adverse event reporting, clinical evaluations including visual function (e.g., visual field), and clinical evaluation.

### Study Design

Patients showing open-angle glaucoma were enrolled in ascending dose groups and received a two intravitreal injections (day 0, day 90) of a peptide having an acetate salt of the structure of Formula III (ONL1204). Visual function metrics were measured at over two screening time points, a baseline, and at about days 90, 177, 180, and 270. Measures for visual function included visual field analysis pre- and post-treatment with ONL1204.

This single-masked, randomized, sham-controlled study of 25 patients was designed to demonstrate the safety of ONL1204 Ophthalmic Solution in patients with progressing open angle glaucoma. Eligible patients were randomized (2:2:1) into 1 of 3 groups (Treatment Groups: ONL1204 Ophthalmic Solution 50 μg dose injection; ONL1204 Ophthalmic Solution 100 μg dose injection; or Sham Group: sham injection) and treated with 2 intravitreal (IVT) injections of 1 of 2 doses of ONL1204 Ophthalmic Solution or 2 sham injections. Patients were part of the study for approximately 39 weeks (270 days). Randomization was be stratified by HVF mean deviation (−5.00 dB to >−10.00 dB and ≤−10.00 dB to −15.00 dB).

### Outcomes

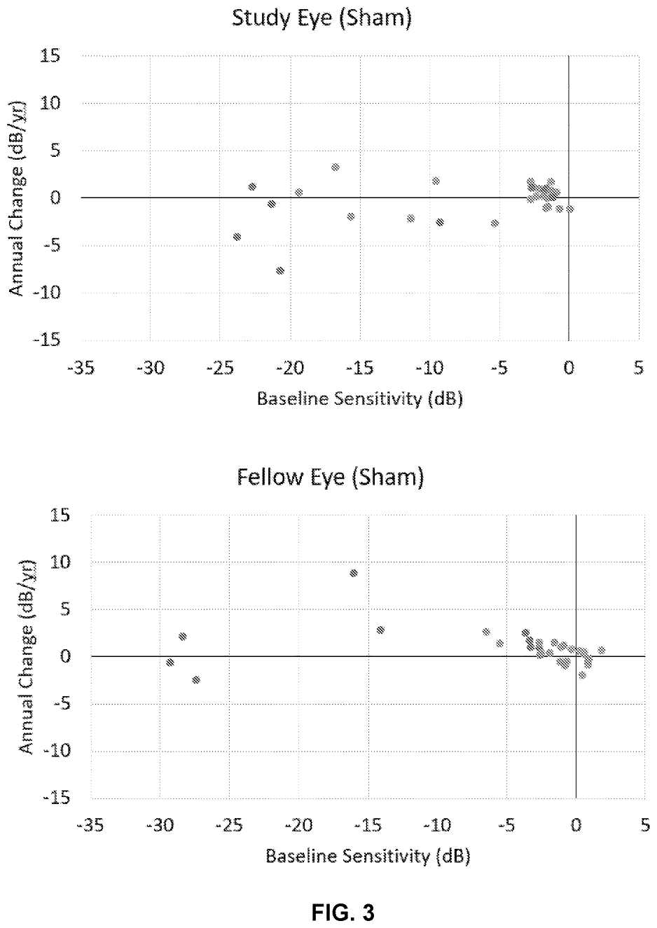

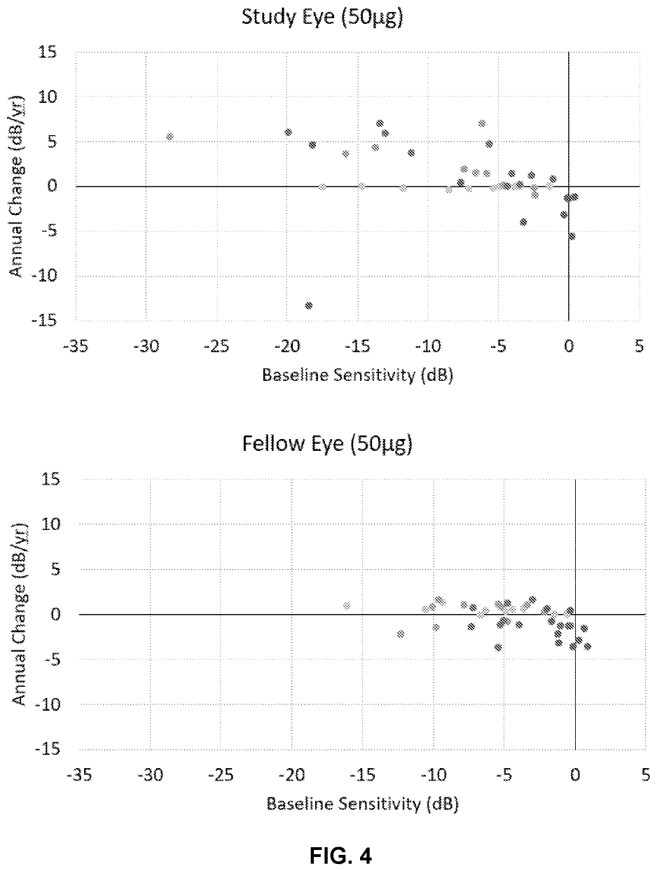

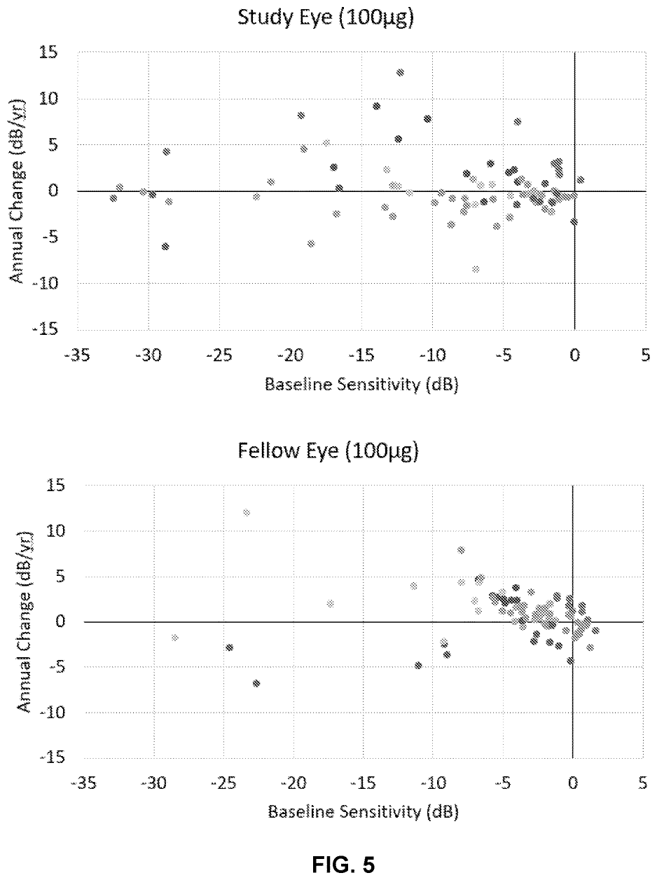

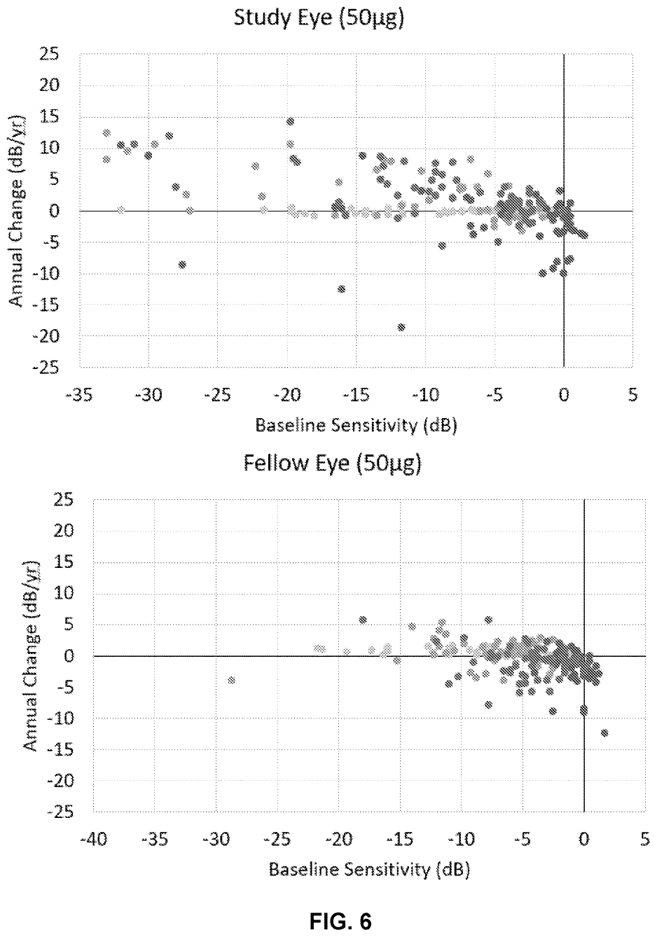

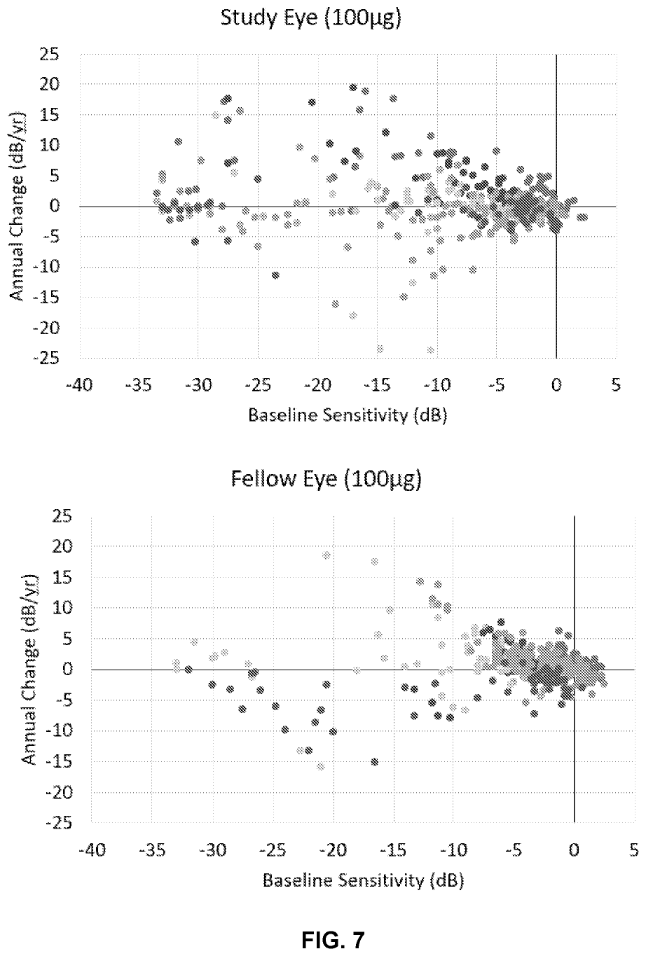

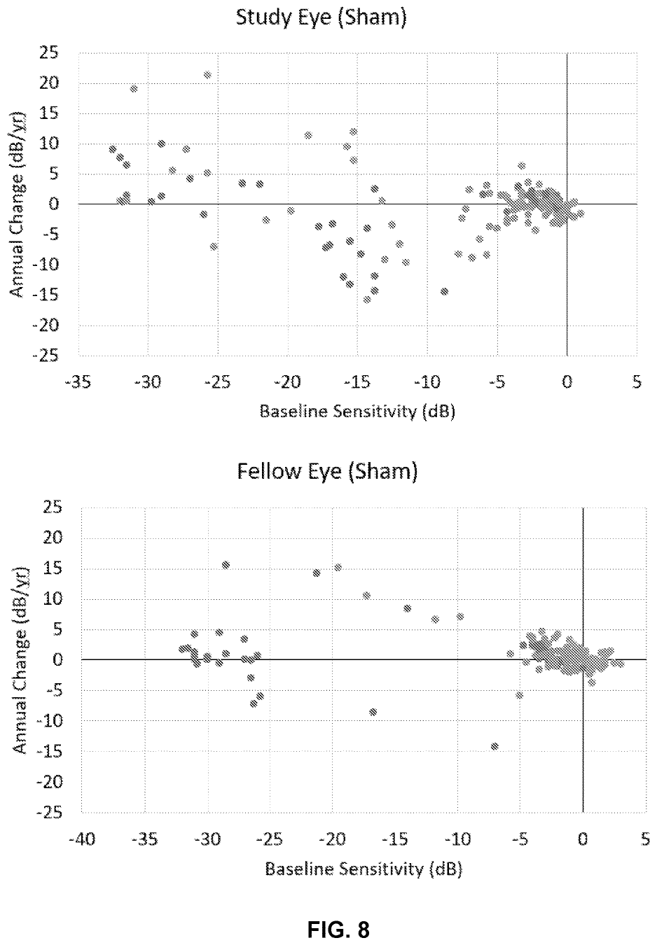

Patients receiving multiple doses comprising 50 ug and 100 ug of ONL1204 showed an improvement in visual field outcomes as measured by Humphrey Visual Field (HVF) analysis that included cluster trend analysis (e.g., measuring changes in local predefined regions/clusters) (see Gardiner et al. Detection of Functional Change Using Cluster Trend Analysis in Glaucoma. Invest Ophthalmol Vis Sci. 2017 May 1). FIGS. 3-5 show study and fellow eye data for the changes in mean visual field analysis of clusters in sham, 50 ug, and 100 ug patients. The data represents mean data for each cluster (10 clusters per patient). X axis (baseline sensitivity) shows the deviation from age-matched normal at baseline, averaged across locations in the cluster. Y Axis (annual change) shows the rate of change total deviation (i.e., change in sensitivity adjusted for normal aging), averaged across locations in the cluster. Each cluster is denoted by a rate of change in sensitivity, in dB/yr. The Baseline sensitivity of each cluster is denoted in dB. Positive values indicate an improvement in visual field and/or improving rate of visual field change. FIGS. 6-8 show study and fellow eye data for the changes in mean visual field analysis (unclustered, point specific, pointwise) in sham, 50 ug, and 100 ug patients.

An improvement in visual field was generally observed for patients receiving ONL1204 as compared to sham. Additionally, the benefits of ONL1204 treatment were also observed in patients having lower baseline sensitivities (e.g., equal to or less than −5).

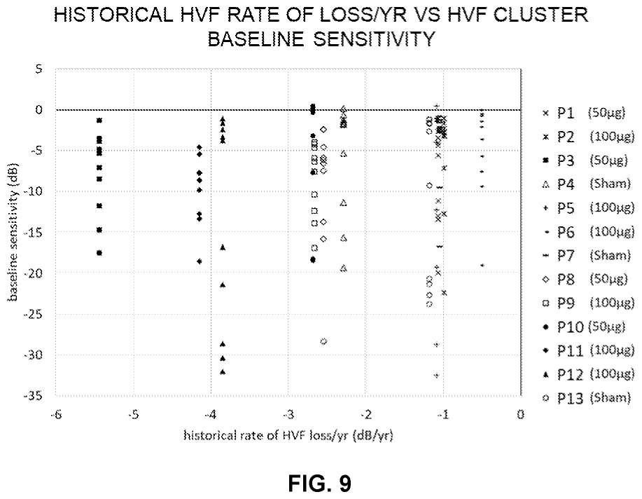

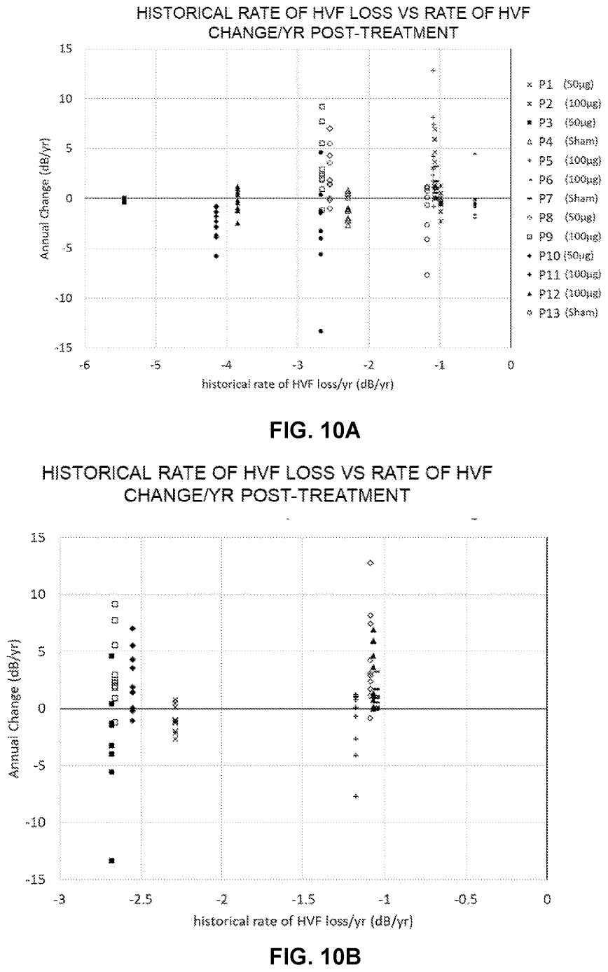

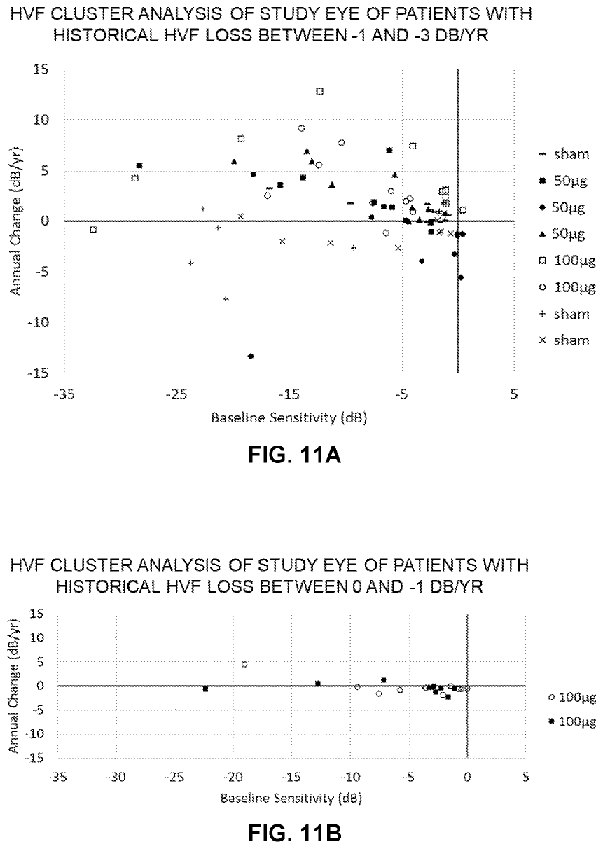

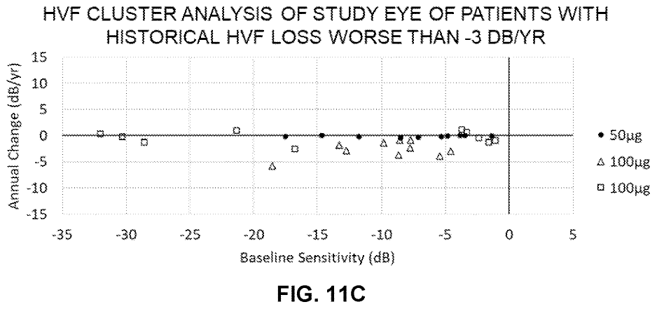

Patient-level analysis of the relationship between historical Humphrey Visual Field (HVF) MD rate of loss/year and Humphrey Visual Field (HVF) baseline sensitivity of clusters of individual points of the visual field is shown in FIG. 9. Treatment with Fas inhibitor ONL1204 generally improved and/or protected the visual field rate of change/year relative to the sham control, across different historical Humphrey Visual Field (HVF) rate of loss/year measurements (e.g., analysis of clusters of points on the visual field as described above). FIGS. 10A and 10B show data from glaucoma patients for historical visual field MD rate of loss/year against visual field rate of change/year of visual field clusters post-treatment with Fas inhibitor ONL1204. FIG. 10B exemplifies patients showing improved HVF and having historical Humphrey Visual Field (HVF) MD rate of loss/year measurements between −1 and −3 dB/year. Treatment with Fas inhibitor ONL1204 generally improved and/or protected against a loss of the visual field rate of change/year relative to the sham control of clusters of the visual field (e.g., analysis of clusters as described above). FIGS. 11A, 11B, and 11C show related data from glaucoma patients for visual field baseline sensitivities against visual field rate of change/year post treatment with Fas inhibitor ONL1204. FIG. 11A shows HVF cluster analysis of study eye of patients with historical HVF loss between −1 and −3 dB/year. FIG. 11B HVF cluster analysis of study eye of patients with historical HVF loss between 0 and −1 dB/year. FIG. 11C shows HVF cluster analysis of study eye of patients with historical HVF loss worse than −3 dB/year.

### Example 2—Improving Visual Outcomes in GA Patients

### Objective

The primary objective was to determine safety of a peptide having the structure of Formula III (ONL1204) and select doses in patients having macular degeneration characterized by geographic atrophy (GA). The primary endpoints were the safety profile demonstrated in this study, including: adverse event reporting, clinical evaluations including best corrected visual acuity (BCVA), and clinical evaluation.

### Study Design

Patients showing GA were enrolled in ascending dose groups and received a single intravitreal injection of a peptide having an acetate salt of the structure of Formula III (ONL1204). Baseline best corrected visual acuity (BCVA) was measured prior to treatment with ONL1204. BCVA was then measured 1, 2-, 4-, 12-, and 24-weeks post treatment with ONL1204. The following doses were evaluated: 50 ug and 100 ug provided in vials with concentrations of 0.5 mg/mL and/or 2 mg/mL of ONL1204.

### Outcomes

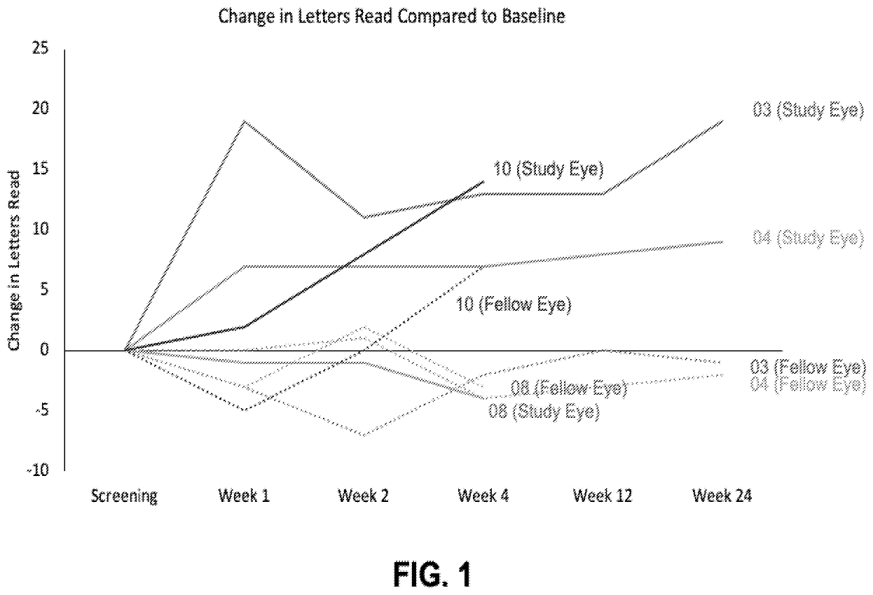

All patients receiving doses comprising 25 ug and 50 ug of ONL1204 showed an improvement in visual outcomes (e.g., visual acuity). FIG. 1 shows study data for the change in best corrected visual acuity (BCVA). An improvement in visual acuity was generally observed for across patients receiving ONL1204, as compared to baseline BCVA (solid lines indicating the study eye and dashed lines indicating the fellow/untreated eye).

### Example 3—Improving Visual Outcomes in Retinal Detachment

### Objective

The primary objective was to determine the Maximum Tolerated Dose (MTD) of a peptide an amino acid sequence HHIYLGAVNYIY and the structure of Formula III (ONL1204) and select doses for further clinical studies. During the study, ocular fluid samples were also collected at the time of study drug injection and at the time of vitrectomy. These samples were used for pharmacokinetic and pharmacodynamic analyses. The primary endpoints were the safety profile demonstrated in this study, including: adverse event reporting, clinical evaluations including best corrected visual acuity (BCVA), and clinical laboratory evaluation. The exploratory endpoints were the pharmacokinetic and pharmacodynamic analyses performed in this study, including: measurement of ONL1204 drug levels in plasma and vitreous fluid for PK analysis, cytokine and other potential molecular biomarker measurement in aqueous and vitreous samples, taken by vitreous tap, and at vitrectomy.

### Study Design

Patients were enrolled in ascending dose groups. After signing informed consent and meeting all eligibility criteria, patients underwent a vitreous tap for cytokine and other potential molecular biomarker analysis followed by a single intravitreal injection of a peptide having an acetate salt of the structure of Formula III (ONL1204). Injection of ONL1204 occurred on the day of diagnosis (Day 1). Safety visits were conducted one day post-injection and as deemed necessary by the examining Investigator (at least 1 visit every 3 days) until Visit 3. At Visit 3 (3+ days post-injection, in accordance with standard of care) patients underwent surgical repair of their retinal detachment with vitrectomy (with or without scleral buckling) and gas or oil tamponade. At the time of surgery, an undiluted vitreous sample was collected for pharmacokinetic and pharmacodynamics analyses, and an aqueous sample was collected for pharmacodynamic analysis. Additional safety visits were targeted for and generally conducted one day post-surgery, 7 days (±3 days) post-surgery, 14 (±4 days) days post-surgery, and Weeks 6, 9, 12 (±7 days each), and Week 24 (±14 days; study exit).

The following doses were evaluated: 25 microgram (ug) and 50 ug provided in vials with concentrations of 0.5 mg/mL and/or 2 mg/mL of Formula III. Therefore, a volume of 0.05 or 0.1 mL was injected into the study eye. Patient participation was approximately 24 weeks (6 months): a single injection treatment and 24 weeks of follow-up.

### Safety

Planned determinations of dose limiting toxicity included consideration of the following events:
1. Ocular inflammation increases by 2 units from pre-injection on the Standardization of Uveitis Nomenclature (SUN) grading scale for aqueous cell or vitreous haze, secondary to inflammation and not the drug formulation itself, identified postinjection of ONL1204 and prior to retinal reattachment surgery;
2. Sustained elevation of IOP characterized as >30 mmHg for 3 consecutive days, postinjection of ONL1204 and prior to retinal reattachment surgery, despite pharmacologic therapy;
3. Reduction in visual acuity from baseline after injection of ONL1204 and prior to retinal reattachment surgery, that in the opinion of the investigator is likely due to the investigational product and results in a: (a) Decrease in visual acuity from 20/200 or 20/400 to light perception without progression of the underlying retinal detachment; or (b) Decrease in visual acuity from baseline to no light perception.
4. Any serious adverse event (SAE) that occurs within the first 14 days following ONL1204 injection that, in the opinion of the investigator, is related to ONL1204.

In total 7 out of 8 patients (4 patients receiving 25 (micrograms (ug) and 4 patients receiving 50 ug of ONL1204) experienced a total of 20 adverse events. Most were mild, and study procedure or surgery related. After evaluating the data for each cohort, the SRC unanimously recommended proceeding with dose escalation to a 100 ug dose.

### Outcomes

All patients receiving doses comprising 25 ug and 50 ug of ONL1204 showed an improvement in visual outcomes (e.g., visual acuity). Table 1 shows study data for Snellen best corrected visual acuity (BCVA). Table 2 shows study data for log MAR best corrected visual acuity (BCVA). As demonstrated by Table 1 and Table 2, an improvement in visual acuity was observed for all patients receiving 25 ug and 50 ug of ONL1204, and across all patient ages within the study. Table 3 shows the time between date of (i) the onset of symptoms or (ii) central vision loss and the date of screening. Table 3 shows the time between the date of (i) the onset of symptoms or (ii) central vision loss and the date of administering ONL1204. As demonstrated by Table 3, improved visual outcomes were observed for all patients receiving ONL1204, even when ONL1204 was administered to patients having greater than 10 days of central vision loss. Table 4 shows the time from administering ONL1204 to performing surgery. Table 4 also shows between the date of (i) the onset of symptoms or (ii) central vision loss and the date of surgery. As demonstrated by Table 4, improved visual outcomes were observed for all patients even when the time from administering ONL1204 to surgery was 3 days or greater (ranging up to 7 days). As demonstrated by Table 4, improved visual outcomes were observed for all patients receiving ONL1204, even when ONL1204 was administered to patients having greater than 15 days of central vision loss prior to the time of surgery to reattach the retina. Table 5 shows the extent (1-12, 12 being full) and height of detachment. As demonstrated by Table 5, improved visual outcomes were observed for all patients receiving ONL1204, across a range of detachment heights and extent of detachments.

Table 6 shows summary statistics for all patients, including sample size (n), mean, and standard deviation (sd). By way of a comparison to surgery alone, a prospective population-based study of 291 Scotland patients with macula-off rhegmatogenous retinal detachments (RRD) without pre-existing retinal disease who had successful repair after one operation reported a baseline mean log MAR visual acuity of 1.71 with follow up mean log MAR visual acuities of 0.87, 0.66, 0.65, and 0.57 at 6 weeks, 3 months, 6 months, and 12 months post-surgery, respectively (Mitry et al. “Long-term visual acuity and the duration of macular detachment: findings from a prospective population-based study” Br J Ophthalmol. 2013 February; 97(2):149-52. doi: 10.1136/bjophthalmol-2012-302330. Epub 2012 Nov. 17. PMID: 23159447). The study further reported for patients having a detachment less than or equal to 8 days a baseline mean log MAR visual acuity of 1.73 with follow up mean log MAR visual acuities of 0.81, 0.60, 0.55, and 0.45 at 6 weeks, 3 months, 6 months, and 12 months post-surgery, respectively. For patients having a detachment greater than 8 days, the study reported a baseline mean log MAR visual acuity of 1.67 with follow up mean log MAR visual acuities of 0.98, 0.82, 0.86, and 0.79 at 6 weeks, 3 months, 6 months, and 12 months post-surgery, respectively.

Table 7 shows the change in cytokine levels from time of ONL1204 administration to surgery. As demonstrated by Table 7, administration of ONL1204 resulted in a mean decrease in MCP1, ICAM, IL1RA, and IL18.

TABLE 1

| | | | BCVA Snellen study eye | | | |
|---|---|---|---|---|---|---|
| Patient ID | Age | Dose (ug) | baseline | week 6 | week 9 | week 24 |
| 001^{#} | 68 | 25 | 20/160 | 20/40 | 20/25 | 20/60 |
| 002^{#} | 62 | 25 | 20/100 | 20/32 | 20/25 | 20/32 |
| 005 | 70 | 25 | 20/200 | 20/125 | 20/100 | 20/100 |
| 002-2* | 70 | 25 | HM | 20/125 | 20/125 | 20/100 |
| 006* | 52 | 50 | CF | 20/50 | 20/50 | 20/50 |
| 007^{%} | 55 | 50 | HM | 20/62.5 | 20/80 | 20/125 |
| 008 | 68 | 50 | CF | 20/50 | — | 20/32 |
| 009^{#%} | 43 | 50 | CF | 20/80 | 20/80 | 20/80 |
| 010*^{%} | 49 | 100 | HM | 20/125 | 20/200 | 20/63 |
| 003 | 56 | 100 | 20/100 | 20/100 | 20/80 | 20/100 |
| 001-2*^{#} | 43 | 100 | 20/200 | 20/200 | HM | 20/400 |
| 002-3*^{%} | 84 | 100 | HM | 20/800 | 20/400 | 20/80 |
| 004-2 | 65 | 200 | 20/160 | 20/50 | 20/40 | 20/32 |
| 012 | 70 | 200 | HM | 20/100 | 20/80 | — |

HM = hand motion
CF = counting fingers
*= received silicone oil replacement,
^{#}= patient demonstrating cataract or potential cataract
^{%}= re-detachment observed in patient
— = data not available

TABLE 2

| | |
|---|---|
| Patient | BCVA logMAR study eye |

| ID | baseline | week 6 | week 9 | week 24 |
|---|---|---|---|---|
| 001 | 0.68 | 0.34 | 0.14 | 0.5 |
| 002 | 0.6 | 0.18 | 0.12 | 0.2 |
| 005 | 1.08 | 0.82 | 0.64 | 0.68 |
| 002-2 | 3 | 0.82 | 0.78 | 0.7 |
| 006 | 2 | 0.38 | 0.34 | 0.34 |
| 007 | 3 | 0.46 | 0.6 | 0.76 |
| 008 | 2 | 0.38 | — | 0.2 |
| 009 | 2 | 0.52 | 0.56 | 0.64 |
| 010 | 3 | 0.8 | 1 | 0.5 |
| 003 | 0.7 | 0.7 | 0.6 | 0.7 |
| 001-2 | 1 | 1 | 3 | 1.3 |
| 002-3 | 3 | 1.6 | 1.4 | 0.6 |
| 004-2 | 0.92 | 0.4 | 0.36 | 0.2 |
| 012 | 3 | 0.7 | 0.6 | — |

(—) = data not available

TABLE 3

| | Time from reported symptom | Time from loss of central |
|---|---|---|
| Patient | onset to administering | vision to administering |
| ID | ONL1204 (days) | ONL1204 (days) |
| 001 | 19 | 15 |
| 002 | 42 | 14 |
| 005 | 22 | 15 |
| 002-2 | 27 | 20 |
| 006 | 14 | 13 |
| 007 | 12 | 12 |
| 008 | 9 | 9 |
| 009 | 28 | 10 |
| 010 | 106 | 12 |
| 003 | 8 | 8 |
| 001-2 | 29 | 10 |
| 002-3 | 9 | 7 |
| 004-2 | 32 | 19 |
| 012 | 12 | 10 |

TABLE 4

| | Time from | Time from reported | Time from loss |
|---|---|---|---|
| | administering | symptom onset | of central |
| Patient | ONL1204 to | of symptoms to | vision to |
| ID | surgery (days) | surgery (days) | surgery (days) |
| 001 | 3 | 22 | 18 |
| 002 | 4 | 46 | 18 |
| 005 | 6 | 28 | 21 |
| 002-2 | 4 | 31 | 24 |
| 006 | 3 | 17 | 16 |
| 007 | 7 | 19 | 19 |
| 008 | 3 | 12 | 12 |
| 009 | 4 | 32 | 14 |
| 010 | 3 | 109 | 15 |
| 003 | 3 | 11 | 11 |
| 001-2 | 6 | 35 | 16 |
| 002-3 | 5 | 14 | 12 |
| 004-2 | 3 | 35 | 22 |
| 012 | 8 | 20 | 18 |

TABLE 5

| Patient | Extent of Detachment | Height of Detachment at |
|---|---|---|
| ID | (1-12, clock hours) | central macula (mm) |
| 001 | 6 | 1 |
| 002 | 11 | 1 |
| 005 | 12 | 1 |
| 002-2 | 7 | 11 |
| 006 | 7 | 5 |
| 007 | 4 | 2 |
| 008 | 5 | 3 |
| 009 | 6 | 4 |
| 010 | 12 | 2 |
| 003 | 3 | 1 |
| 001-2 | 7 | — |
| 002-3 | 6 | — |
| 004-2 | 7 | 1 |
| 012 | 7 | 2 |

(—) = data not available

TABLE 6

| | n | mean | sd |
|---|---|---|---|
| Baseline | 14 | 1.86 | 0.99 |
| Week 6 | 14 | 0.65 | 0.36 |
| Week 9 | 13 | 0.78 | 0.74 |
| Week 24 | 13 | 0.56 | 0.30 |

TABLE 7

| | |
|---|---|
| | Percent Change (%) from time of |
| Patient | administering ONL1204 to surgery |

| ID | MCP1 | ICAM1 | IL1RA | IL18 |
|---|---|---|---|---|
| 001 | −41.4 | −82.4 | −51.0 | −60.5 |
| 002 | −6.02 | −42.7 | −57.4 | −22.5 |
| 002-2 | −35.9 | −86.2 | −99.3 | −50.8 |
| 006 | 11.6 | 0 | −88.3 | 0 |
| 007 | −3.12 | 64.0 | −60.0 | 0 |
| 008 | −48.4 | −100 | −94.4 | −100 |
| 010 | −9.5 | −43.9 | 8.5 | −33.7 |
| 003 | 78.5 | 24.9 | −46.1 | 0 |
| 001-2 | 500 | 22.7 | −65.2 | 66.0 |
| 004-2 | −2.4 | 0 | −26.3 | 0 |

data not available or adverse event not related to drug was observed for Patient ID 005, Patient ID 009, Patient ID 002-3, and Patient ID 012

### Example 4—Pharmacokinetics Studies in Rabbit Eye

### Objective

A study was performed to evaluate the ocular tissue concentration of a peptide having an acetate salt of the structure of Formula III (ONL1204) up to 168 days following a single intravitreal injection to both eyes and a repeat intravitreal injection on Day 90 in a select group. A total of 33 rabbits were evaluated on study to evaluate ONL1204 over 168 days.

### Study Design

Animals were observed for any abnormal observations prior to dosing, approximately weekly (for surviving animals through Week 12), and at euthanasia. Body weights were measured and recorded prior to first dose, approximately weekly (for surviving animals through Week 12), and prior to euthanasia. No ONL1204-related effects on body weights or clinical observations were evident during the study. Ophthalmic examinations were performed using the Hackett-McDonald Scoring System on Days 0, 7, 14, 28, 90, 97, 151, and 168. Pharmacokinetic analyses were performed on the vitreous humor concentration data.

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5).

Vitreous humor, retina, and choroid were collected on days 1, 7, 14, 28, 42, 61, 90, 97, 151, and 168. Eyes were enucleated, snap frozen using liquid nitrogen, and kept on dry ice or frozen at −80° C. until dissected for collection of aqueous humor, vitreous humor, retina, choroid, iris-ciliary body (ICB), and lens.

### Sample Preparation

To homogenize ocular tissue samples, weighed amounts of control bovine vitreous humor, retina, and choroid were homogenized in USA scientific impact resistant microtubes containing 2.8 mm ceramic beads. Unknown rabbit vitreous humor, retina, and choroid samples were homogenized in USA scientific impact resistant microtubes containing 2.8 mm ceramic beads. Each vitreous humor sample was homogenized after the addition of 5000 microliters (uL) diluent (acetonitrile:water: 1 M hydrochloric acid (70:20:10, v/v/v). Retina tissues were homogenized after the addition of diluent (IPA:NH4OH (1000:1, v/v)) at a 1:5 ratio (parts tissue to parts diluent), and choroid samples were homogenized following addition of diluent at a 1:19 ratio. Tissues were homogenized at 5500 rpm for 3×30 second cycles with 20 second pauses between cycles until homogenized. Choroid went through two runs of homogenization, and retina and vitreous humor samples went through one run of homogenization.

Preparation of Calibration Stock and Working Standards: A stock calibration standard was prepared in dimethylsulfoxide (DMSO) at a concentration of 500 ug/mL for ONL1204. Working calibration standards were prepared for vitreous humor by serial dilution of working stock solution with acetonitrile:water:formic acid (25:75:0.1, v/v/v) over a range of 500 ng/mL to 500,000 ng/mL ONL1204. Working calibration standards were prepared for choroid and retina by serial dilution of working stock solution with acetonitrile:water:formic acid (25:75:0.1, v/v/v) over a range of 5.00 ng/mL to 1,000 ng/mL ONL1204.

Preparation of Standards, Unknowns, Blanks, and Blanks with Internal Standard for Vitreous Humor Analysis: In a polypropylene tube, 10 uL of working calibration standard was added to 90 uL control blank vitreous humor. For blanks and blanks with internal standard, 100 uL of control blank bovine vitreous humor was added. Five hundred (500) uL of acetonitrile:water:1M hydrochloric acid (70:20:10, v/v/v) was added to each standard and 500 uL of acetonitrile:water:1M hydrochloric acid (70:20:10, v/v/v) was added to blanks. Standards and blanks were vortex mixed. One hundred (100) uL of each standard, blank or unknown vitreous humor sample homogenate was then aliquoted. One hundred (100) uL of IPA:NH4OH (1000:1, v/v) was added to each sample. The samples were vortex mixed for 1 minute, then centrifuged for 10 minutes at 4,000 rpm (4° C.). In an autosampler plate, 50.0 uL of working internal standard (WIS) (5,000 ng/mL APi1887 in acetonitrile:water:formic acid (25:75:0.1, v/v/v)) was added to 50.0 uL supernatant. Fifty (50.0) uL of acetonitrile:water:1M hydrochloric acid (70:20:10, v/v/v) was added to blank without IS. Two hundred (200) uL of water was added. The samples were then mixed using a multichannel pipette, and supernatant transferred to an autosampler plate for analysis.

Preparation of Standards, Unknowns, Blanks, and Blanks with Internal Standard for Retina and Choroid: In a 96-well plate, 100 uL of control retina or choroid homogenate was added for calibration samples and unknowns. For blanks and blanks with internal standard, 100 uL of control bovine retina or choroid tissue homogenate was added. Twenty (20) uL of WIS (5,000 ng/mL APi1887 in acetonitrile:water:formic acid [25:75:0.1, v/v/v]) was added to each calibration standard and blank with IS sample. Twenty (20) uL acetonitrile:water:formic acid (25:75:0.1, v/v/v) was added to each blank without IS sample. Three-hundred (300) uL of IPA:NH4OH (1000:1, v/v) was added to each choroid or retina sample. The samples were then vortex mixed for 5 minutes. The samples were allowed to set for 15 minutes, vortex mixed for an additional minute, and centrifuged for 10 minutes at 4,000 rpm (4° C.).

Three-hundred-fifty (350) uL of each sample was then aliquoted to a 96-well plate. The samples were dried with nitrogen at 35° C. Two-hundred (200) uL of acetonitrile:water:formic acid (25:75:0.1, v/v/v) was added to reconstitute each sample. The samples were then vortex mixed for 4 minutes, and transferred to a 96-well autosampler plate for analysis.

Based on the results, the t½ for animals receiving a single dose was 279.8 days (0.1 mg/eye, days 0 to 168). Table 8 shows drug concentrations in the vitreous humor and retinal issue. ONL1204 was detected in the vitreous humor across all time points analyzed. Retinal tissue is generally difficult to assay, wherein detection in the retina represents the minimal amount of ONL1204 present (i.e., the values do not likely overestimate the concentration of ONL1204 in retinal tissue (limit of quantification (LLOQ)=0.500 ng/mL, 3.00 ng/g for the retina, unless marked with “<LLOQ*” which indicates LLOQ=1.00 ng/mL, 6.0 ng/g; ** denotes animal group receiving a second injection at day 90). Nonetheless, ONL1204 was detected in retinal tissue as well as the vitreous humor after 168 days.

TABLE 8

| | |
|---|---|
| Sample | Conc. of ONL1204 |

| ID** | Day | Vitreous Humor (ug) | Retina (ng/g) |
|---|---|---|---|
| 1A-Right | 1 | 76 | <LLOQ |
| 1A-Left | 1 | 48.3 | 12.7 |
| 1B-Right | 1 | 80.9 | <LLOQ |
| 1B-Left | 1 | 89.6 | <LLOQ |
| 1C-Right | 1 | 74.9 | <LLOQ |
| 1C-Left | 1 | 77.1 | <LLOQ |
| 2A-Right | 7 | 83 | <LLOQ |
| 2A-Left | 7 | 41.6 | <LLOQ |
| 2B-Right | 7 | 82.6 | <LLOQ |
| 2B-Left | 7 | 83.6 | <LLOQ |
| 2C-Right | 7 | 79.2 | <LLOQ |
| 2C-Left | 7 | 67.8 | 85.2 |
| 3A-Right | 14 | 67.3 | <LLOQ |
| 3A-Left | 14 | 67.4 | <LLOQ |
| 3B-Right | 14 | 54.2 | <LLOQ |
| 3B-Left | 14 | 70.9 | <LLOQ |
| 3C-Right | 14 | 63.2 | 5.95 |
| 3C-Left | 14 | 74.5 | <LLOQ |
| 4A-Right | 28 | 76.9 | <LLOQ |
| 4A-Left | 28 | 70.2 | <LLOQ |
| 4B-Right | 28 | 74.4 | <LLOQ |
| 4B-Left | 28 | 82 | <LLOQ |
| 4C-Right | 28 | 69.9 | <LLOQ |
| 4C-Left | 28 | 63.9 | <LLOQ |
| 5A-Right | 42 | 71.7 | <LLOQ |
| 5A-Left | 42 | 69.3 | <LLOQ |
| 5B-Right | 42 | 65.3 | <LLOQ |
| 5B-Left | 42 | 75.7 | <LLOQ |
| 5C-Right | 42 | 60.9 | <LLOQ |
| 5C-Left | 42 | 62.3 | <LLOQ |
| 6A-Right | 61 | 54.8 | <LLOQ |
| 6A-Left | 61 | 55.2 | <LLOQ |
| 6B-Right | 61 | 58.3 | <LLOQ |
| 6B-Left | 61 | 54.5 | <LLOQ |
| 6C-Right | 61 | 59.9 | <LLOQ |
| 7A-Right | 90 | 63.7 | <LLOQ* |
| 7A-Left | 90 | 61.5 | <LLOQ* |
| 8A-Right | 90 | 54.1 | <LLOQ* |
| 8A-Left | 90 | 51 | <LLOQ* |
| 7B-Right** | 97 | 131 | 9.55 |
| 7B-Left** | 97 | 135 | <LLOQ* |
| 8B-Right** | 97 | 127 | 8.52 |
| 8B-Left** | 97 | 113 | <LLOQ* |
| 8C-Right** | 151 | 99.1 | <LLOQ* |
| 8C-Left** | 151 | 103 | 15.6 |
| 9A-Right** | 151 | 113 | 52.9 |
| 9A-Left** | 151 | 124 | 9.54 |
| 9B-Right | 168 | 99.8 | 83.9 |
| 9B-Left | 168 | 103 | <LLOQ* |
| 9C-Right | 168 | 75.9 | <LLOQ* |
| 9C-Left | 168 | 85.6 | <LLOQ* |
| 10B-Right | 168 | 48.7 | 32.9 |
| 10B-Left | 168 | 45.7 | 8.4 |
| 10C-Right | 168 | 55.8 | <LLOQ* |
| 10C-Left | 168 | 44.7 | <LLOQ* |

Right or left designates the right or left eye, respectively.

### Example 5—Sodium Iodate (NaIO₃) Challenge Study

### Objective

The objective of this study was to test the duration of activity (e.g., protection) of ONL1204 in the male rabbit NaIO₃ model by treating animals with test article (ONL1204) on Day 0 and staggering the day of NaIO₃ insult to assess duration of protection offered by ONL1204. Demonstrating a protective effect of ONL1204 at extended timepoints would support the use of ONL1204 ophthalmic solution in chronic ophthalmic diseases.

### Study Design

Test Article: Test Article: ONL 1204 Formulation; Administration Route: 50 ul by IVT injection; Frequency of Administration: Single dose on Day 0; Formulations: Group 1-4; 9: 2 mg/ml ONL 1204 in 4.5% mannitol, 0.4% poloxamer 407, 10 mM acetate buffer pH 4.5, Group 5-8: 0.5 mg/ml ONL 1204 in 4.5% mannitol, 0.4% poloxamer 407, 10 mM acetate pH 4.5; Dose: Group 1-4; 9: 100 μg per eye, Group 5-8: 25 μg per eye

NaIO₃ solution: Challenge Article: NaIO₃; Administration Route: IV; Frequency of Administration: Day 4 (day 7 endpoint), 14 (day 17 endpoint), 28 (day 31 endpoint), 59 (day 61 endpoint), or 78 (day 81 endpoint (rechallenged group)). Solution: 20 mg/ml in 0.9% Injectable Saline; Dose: 17 or 18 mg/kg body weight

Test System: Species: Rabbit; Expected Age: Animals were between 3 and months of age; Expected Body Range: Approximately 2.0 Kg, Identification Animals were individually housed and identified via cage card and ear ID and/or ear tattoo.

Intravitreal Injection: On Day 0, eyes were anesthetized with proparacaine and pupils dilated with tropicamide. Animals were anesthetized with isoflurane vapors to effect. A few drops of Betadine were placed on the eye and then rinsed with ophthalmic eye wash. A clean speculum was placed in the eye (rinsed in Betadine and sterile saline) and the temporal sclera was marked with calipers 3.5 mm from the corneal border. A BD Ultra-Fine 31 g, 3/10 cc, 5/16 in, Insulin Syringe with 50 ul of test article of 0.5 mg/ml or 2.0 mg/ml ONL1204 was inserted though indentation into the mid-vitreous approximately 62° from the horizontal axis (280 from the vertical axis) and test article was injected into the vitreous. In order to reduce the potential for reflux of the test article the needle remained in the vitreous for at least 10 seconds following injection. No reflux was noted on the dosing record.

Model Induction: A 20 mg/ml solution of NaIO₃ was prepared in 0.9% injectable saline aliquoted into 4 batches and frozen at −20° C. For Groups 1-8, 1 batch of NaIO₃ was thawed prior to injection. Groups 9-10 were added after the Study was underway therefore a new solution of 20 mg/ml NaIO₃ was prepared in 0.9% injectable saline and frozen at −20° C. and thawed prior to injection. Test article and NaIO₃ were injected into the left ear.

Animals were weighed and anesthetized with isoflurane vapors to effect. The left ear was shaved and swabbed with 10% alcohol prior to injection. An indwelling catheter was placed in the left marginal ear vein and flushed with 0.9% injectable saline. Animals were weighed just prior to dosing and Groups 1-8 received 18 mg NaIO₃/kg bodyweight followed by a flush of 0.3 ml 0.9% injectable saline. Groups 9-10 received 16 mg NaIO₃/kg bodyweight followed by a flush of 0.3 ml 0.9% injectable saline. All injections were performed without complication and no reflux or leakage was noted.

Fluorescein Angiography: Eyes were anesthetized with proparacaine and pupils dilated with tropicamide and phenylephrine. Animals were anesthetized with isoflurane vapors to effect. 50 ul of 25% sodium fluorescein was injected into the marginal ear vein and allowed to circulate for at least 2 minutes prior to imaging. In animals with intact retinal pigment epithelial cell pigmentation, it is not possible to stimulate or visualize the light emitted by fluorescein circulating in the choroidal vasculature. NaIO₃ causes one or more regions of retinal pigment epithelial cell death and depigmentation which creates a window into the choroid. Images were captured to best map the full extent of visible choroid/retinal pigment epithelial cell death in both eyes.

Data Analysis: The area of RPE depigmentation is reported as a unitless value provided by the ImageJ software. In certain cases where the damage extended well into the periphery it was not possible to image the entire edge of the affected area. In some cases, a typical lesion was not created in either eye. In these cases, it is possible to measure the accumulation of lipofuscin, the phagocytized shed outer segments normally processed by the RPE, which builds up in unhealthy RPE. In these cases, ImageJ was used to highlight the autofluorescence around the Optic Nerve Head by thresholding the image with a 100/20 window level. The resulting area was then measured.

### Results

The mean lesion size in both eyes of 3 male rabbits, N=6, were used as negative control, against which the protective effect of ONL1204 treatment was compared. On Day 4, there were equally small lesions in both treatment groups and no statistical difference in lesion area. Both Groups were significantly lower than negative control. On Day 14, animals treated with 100 ug had significantly smaller lesion size then both negative controls and those treated with 25 ug. On Day 28, animals treated with 100 ug had significantly smaller lesion size then both negative control animals and those treated with 25 ug. A substantive reduction in mean lesion size was observed across challenge time points. On Day 59, the mean lesion size for animals treated with 25 ug and 100 ug was 40% and 50% smaller respectively compared to negative controls. Results are shown in FIG. 1.

### Example 6—Pharmacokinetics Studies in Minipig

### Objective

A study was performed in minipig to evaluate the ocular tissue concentrations of a peptide having the structure of Formula III (ONL1204) following a single intravitreal injection.

### Study Design

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5). Vitreous humor, retina, and choroid were collected on days 1, 7, 14, 28, and 88.

### Pharmacokinetics

Table 9 shows drug concentration results in the vitreous humor, retinal issue, and choroid. ONL1204 was detected in the vitreous humor across all time points analyzed. Due to assay limitations, detection in the retina represents the minimal amount of ONL1204 present (i.e., the values do not likely overestimate the concentration of ONL1204 in retinal tissue) (limit of quantification (LLOQ) for vitreous humor=0.5 ug/mL; LLOQ for retina=0.500 ng/mL, 2.5 ng/g unless noted by “<LLOQ*” indicating LLOQ=1.00 ng/mL, 5.0 ng/g; LLOQ for choroid=1.00 ng/mL, 20.0 ng/g).

TABLE 9

| | | | Concentration | | |
|---|---|---|---|---|---|
| Animal | Dose | | Vitreous humor | Retina | Choroid |
| ID | (ug/eye) | Day | (μg) | (ng/g) | (ng/g) |
| 1 | Vehicle | 6 | <LLOQ | <LLOQ | <LLOQ |
| 2 | | 6 | <LLOQ | <LLOQ | <LLOQ |
| 3 | | 6 | <LLOQ | <LLOQ | <LLOQ |
| 4 | | 28 | <LLOQ | <LLOQ | <LLOQ |
| 5 | | 28 | <LLOQ | <LLOQ | <LLOQ |
| 6 | | 28 | <LLOQ | <LLOQ | <LLOQ |
| 7 | | 88 | <LLOQ | <LLOQ* | <LLOQ |
| 8 | | 88 | <LLOQ | <LLOQ* | <LLOQ |
| 9 | | 88 | <LLOQ | <LLOQ* | <LLOQ |
| 10 | 200 | 6 | 130 | <LLOQ | 32.4 |
| 11 | | 6 | 106 | 4.16 | 352 |
| 12 | | 6 | 111 | 49.3 | 182 |
| 13 | | 28 | 156 | 6.14 | 166 |
| 14 | | 28 | 133 | <LLOQ | <LLOQ |
| 15 | | 28 | 107 | <LLOQ | 33.5 |
| 16 | | 88 | 95.1 | <LLOQ* | 31 |
| 17 | | 88 | 75.5 | <LLOQ* | 62.5 |
| 18 | | 88 | 54.0 | <LLOQ* | 21 |
| 19 | 300 | 6 | 205 | 9.5 | <LLOQ |
| 20 | | 6 | 141 | 12.1 | 125 |
| 21 | | 6 | 153 | <LLOQ | 178 |
| 22 | | 28 | 124 | 39.3 | 77.7 |
| 23 | | 28 | 232 | 25.5 | 119 |
| 24 | | 28 | 184 | 6.59 | 50.5 |
| 25 | | 88 | 88.3 | <LLOQ* | <LLOQ |
| 26 | | 88 | 137 | <LLOQ* | 89.4 |
| 27 | | 88 | 99.6 | <LLOQ* | <LLOQ |

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

SEQUENCES

| SEQ ID NO | SEQUENCE |
|---|---|
| 1 | HHIYLGAVNYIY |
| 2 | YIYNVAGLYIHH |

1. A method of improving visual function (e.g., BCVA or visual field) in an eye of an individual having an ocular disease or disorder, comprising:
(a) administering a first composition comprising a peptide to the eye of the individual, the peptide comprising an amino acid sequence HHIYLGAVNYIY or a variant sequence thereof, or a pharmaceutically acceptable salt thereof; and
(b) administering a second composition comprising the peptide to the eye, wherein the second composition is administered about 10 weeks or more after administering the first composition.

2. The method of claim 1, wherein the method comprises administering the second composition about 11 weeks to about 13 weeks after the first administering the first composition.

3. The method of any one of claims 1 to 2, wherein the method comprises administering the first composition and the second composition to the vitreous humor of the eye.

4. A method of improving visual function (e.g., BCVA or visual field) in an eye of an individual having an ocular disease or disorder, comprising:
administering a plurality of compositions to the eye, wherein:
each composition of the plurality of compositions comprises a peptide, the peptide comprising an amino acid sequence HHIYLGAVNYIY or variant sequence thereof, or a pharmaceutically acceptable salt thereof; and
no greater than five compositions are administered to the eye within a year.

5. The method of claim 4, wherein the method comprises administering each composition of the plurality of compositions to the vitreous humor of the eye.

6. The method of any one of claims 1 to 5, wherein the peptide has a half-life in the vitreous humor of (e.g., at least) about 30 to 300 days.

7. The method of claim 6, the peptide has a half-life in the vitreous humor of (e.g., at least) about 90 to 300 days.

8. The method of any one of claims 1 to 7, wherein the method comprises using the vitreous humor as a depot to provide the peptide to retinal tissue in the eye, wherein the peptide is present in the vitreous humor for about 30 to 365 days after administration.

9. The method of any one of claims 1 to 8, wherein the peptide is provided to the retina from the vitreous humor for at least about 30 to 365 days after administration.

10. The method of any one of claims 1 to 9, wherein the peptide is present (e.g., detectable) in the vitreous humor at about 90 to 365 days after administration.

11. The method of any one of claims 1 to 10, wherein the ocular disease or disorder comprises inflammation in retinal tissue and/or a symptom thereof.

12. The method of any one of claims 1 to 11, wherein the ocular disease, disorder, or condition comprises photoreceptor cell death and/or a symptom thereof.

13. The method of any one of claims 1 to 12, wherein the ocular disease or disorder comprises retinal degeneration and/or a symptom thereof.

14. The method of any one of claims 1 to 13, wherein the ocular disease or disorder comprises a loss and/or decrease in visual acuity.

15. The method of any one of claims 1 to 14, wherein the ocular disease or disorder comprises macular degeneration and/or a symptom thereof.

16. The method of claim 15, wherein the macular degeneration comprises geographical atrophy.

17. The method of claim 15 or 16, wherein the macular degeneration is age-related macular degeneration.

18. The method of any one of claims 1 to 14, wherein the ocular disease or disorder comprises glaucoma and/or a symptom thereof.

19. The method of claim 18, wherein in the glaucoma comprises open angle glaucoma.

20. The method of claim 19, wherein the glaucoma is progressing glaucoma.

21. The method of any one of claims 1 to 20, wherein the variant sequence comprises an amino acid substitution.

22. The method of claim 21, wherein the variant sequence comprises one amino acid substitution.

23. The method of claim 21, wherein the variant sequence comprises two amino acid substitutions.

24. The method of any one of claims 1 to 23, wherein the peptide further comprises a modification.

25. The method of claim 24, wherein the modification comprises a modified amino acid.

26. The method of any one of claims 1 to 25, wherein the peptide comprises an amidated C-terminus.

27. The method of any one of claims 1 to 26, wherein the peptide has the structure of Formula I or a pharmaceutically acceptable salt thereof.

28. The method of any one of claims 1 to 27, wherein the peptide has the structure of Formula III: 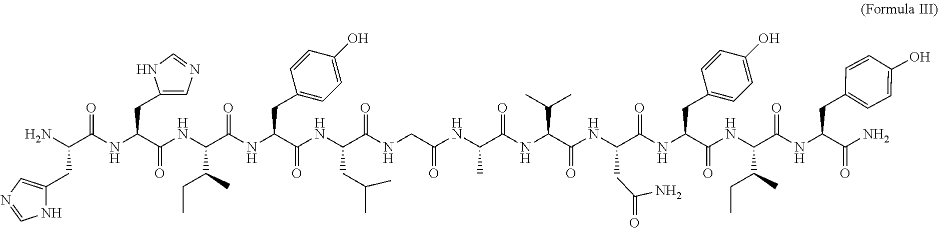 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]
or a pharmaceutically acceptable salt thereof.

29. The method of any one of claim 1 to 28, wherein the composition (e.g., each composition or the first/second composition) comprises the pharmaceutically acceptable salt of the peptide.

30. The method of claim 29, wherein the pharmaceutically acceptable salt is an acetate salt.

31. The method of claim 30, wherein the pharmaceutically acceptable salt is a polyacetate salt.

32. The method of claim 31, wherein the polyacetate salt is a triacetate salt.

33. The method of claim 30, wherein the pharmaceutically acceptable salt is a hydrochloride salt.

34. The method of any one of claims 1 to 33, wherein the composition (e.g., each composition or the first/second composition) further comprises one or more excipients.

35. The method of any one of claims 1 to 33, wherein the composition (e.g., each composition or the first/second composition) further comprises a surfactant.

36. The method of claim 35, wherein the surfactant is a non-ionic surfactant.

37. The method of claim 36, wherein the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer.

38. The method of claim 36 or 37, wherein the surfactant forms about 0.01% to about 20% weight/weight of the composition.

39. The method of claim 38, wherein the surfactant forms about 0.05% to about 10% weight/weight of the composition.

40. The method of any one of claims 1 to 39, wherein the composition (e.g., each composition or the first/second composition) further comprises a tonicity adjusting agent, a buffering agent, or a combination thereof.

41. The method of any one of claims 1 to 40, wherein the composition is buffered at a pH of 2.5 to 7.5.

42. The method of any one of claims 1 to 41, wherein the composition comprises 50 micrograms (ug) to 500 ug of the peptide.

43. The method of any one of claims claim 1 to 42, wherein the peptide is present at a concentration 0.1 milligrams per milliliter (mg/mL) to 10.0 mg/mL.
